(19) United States
(12) Patent Application Publication
(10) Pub. No.: US 2023/0002756 A1
(43) Pub. Date: Jan. 5, 2023

(54) High Performance Platform for Combinatorial Genetic Screening

(71) Applicant: THE TRUSTEES OF THE UNIVERSITY OF PENNSYLVANIA, Philadelphia, PA (US)

(72) Inventors: Junwei Shi, Philadelphia, PA (US); Rodrigo Gier, Lansdowne, PA (US)

(21) Appl. No.: 17/784,645

(22) PCT Filed: Dec. 11, 2020
(86) PCT No.: PCT/US2020/064523

§ 371 Date: Jun. 11, 2022

Related U.S. Application Data
(60) Provisional application No. 62/947,479, filed on Dec. 12, 2019.

(51) Int. Cl. C12N 15/10; C12Q 1/6886

(52) U.S. Cl. CPC C12N 15/1034; C12N 15/102; C12Q 1/6886; C12N 2310/20; C12Q 2600/106

(57) ABSTRACT

The current invention provides methods of combinatorial genetic screening in a cancer cell comprising an enhanced CRISPR-Cas12a system, and compositions comprising the same. Also provided are methods for screening for synergistic combinations of drug targets, as well as the treatment of cancer in a subject in need thereof.

### CROSS-REFERENCE TO RELATED APPLICATION

The present application is entitled to priority under 35 U.S.C. § 119(e) to U.S. Provisional Patent Application No. 62/947,479, filed Dec. 12, 2019 which is hereby incorporated by reference in its entirety herein.

### BACKGROUND OF THE INVENTION

Unbiased forward genetic screening approaches have been widely used for new cancer drug target discovery. To date, most screens have been limited to identifying cancer growth dependencies at the single-gene level. While significant, these screens only capture a small subset of potential targets, completely missing synthetic sick/lethal genetic interactions that arise only from the simultaneous perturbation of two or more genes. Combinatorial genetic screening, a high-throughput strategy to perturb multiple genes simultaneously, holds great promise for discovering therapeutically tractable synthetic sick/lethal interactions across diverse disease types, including cancers. Currently, Cas9-based methods are efficient in single-gene knockout screening, but underperform in combinatorial genetic screening, primarily due to the high recombination frequency and complicated cloning of the dual CRISPR-Cas9 RNA (sgRNA) expression vector. Cas12a (formerly Cpf1), an RNA-programmable DNA endonuclease similar to Cas9, has the potential to overcome these limitations. Unlike Cas9, Cas12a has the intrinsic RNase activity to process its own crRNA array, enabling multi-gene editing from a single RNA transcript.

There is a great need in the art for simple and robust CRISPR-based combinatorial genetic screening techniques in cancer cells. The present invention addresses this need.

### SUMMARY OF THE INVENTION

As described herein, the present invention relates to methods of combinatorial genetic screening comprising administering an enhanced variant of a CRISPR-Cas12a system to target cells. Also provided are compositions comprising said enhanced variant of a CRISPR-Cas12a system, as well as methods for identifying synergistic combinations of drug targets and treating cancer in a subject in need thereof.

In one aspect, the invention includes a method of combinatorial genetic screening in a cancer cell, the method comprising administering to the cancer cell a CRISPR-Cas12a system, wherein the CRISPR-Cas12a system comprises an enhanced Cas12a variant, a modified nuclear localization signal (NLS), and a modified dual direct repeat CRISPR RNA (crRNA), whereby the CRISPR-Cas12a system mutates multiple genomic regions simultaneously, and combinatorial genetic screening is achieved.

In certain embodiments, the enhanced Cas12a variant is an Acidaminococcus Cas12a (AsCas12a) variant.

In certain embodiments, the AsCas12a comprises a E174R mutation and/or a S542R mutation.

In certain embodiments, the enhanced Cas12a variant has increased DNA binding affinity and activity.

In certain embodiments, the modified NLS comprises six copies of a NLS.

In certain embodiments, the modified crRNA comprises from 3′ to 5′ a first direct repeat (DR) sequence, a gRNA, and a second direct repeat sequence.

In certain embodiments, the crRNA comprises from 3′ to 5′ a first direct repeat sequence, a first gRNA, a second direct repeat sequence, a second gRNA, and a third direct repeat sequence.

In various embodiments of the above aspect or any other aspect or embodiment of the present invention, any one of the direct repeats is about 19 nucleotides in length.

In certain embodiments, the screening identifies genomic regions involved in cancer pathogenesis.

In certain embodiments, the screening identifies epigenetic interactions in the cancer cell.

In certain embodiments, the epigenetic interactions are synthetic sick/lethal interactions.

In certain embodiments, the methods of the invention further comprise designing a cancer treatment based on the screening results.

In certain embodiments, the methods of the invention further comprise a crRNA library, wherein the crRNA library comprises a plurality of crRNAs targeting a plurality of genomic regions involved in epigenetic regulation.

In certain embodiments, each crRNA in the crRNA library comprises from 3′ to 5′ a first direct repeat sequence, a first gRNA, and a second direct repeat sequence.

In certain embodiments, each crRNA in the crRNA library comprises from 3′ to 5′ a first direct repeat sequence, a first gRNA, a second direct repeat sequence, a second gRNA, and a third direct repeat sequence.

In another aspect, the invention includes a method of identifying a combination of drug targets wherein a therapeutically synergistic effect is elicited when the combination of targets is treated, the method comprising:
administering to a cell a composition comprising a CRISPR-Cas12 system, wherein the CRISPR-Cas12a system comprises an enhanced Cas12a variant, a modified nuclear localization signal (NLS), and a modified dual direct repeat CRISPR RNA (crRNA),
whereby the CRISPR-Cas12a system mutates multiple genomic regions simultaneously, thereby identifying the drug target combination.

In another aspect, the invention includes a method of treating cancer in a subject in need thereof, the method comprising:
administering a CRISPR-Cas12a system to a cancer cell from the subject,
wherein the CRISPR-Cas12a system comprises an enhanced Cas12a variant, a modified nuclear localization signal (NLS), and a modified dual direct repeat CRISPR RNA (crRNA),
whereby the CRISPR-Cas12a system mutates multiple genomic regions simultaneously, and combinatorial genetic screening is performed,
determining a cancer treatment based on the screening results, and
administering the cancer treatment to the subject.

In various embodiments of the above aspects or any other aspect of the present invention, the enhanced Cas12a variant is an Acidaminococcus Cas12a (AsCas12a) variant.

In certain embodiments, the AsCas12a comprises a E174R mutation and/or a S542R mutation.

In various embodiments of the above aspects or any other aspect of the present invention, the enhanced Cas12a variant has increased DNA binding affinity and activity.

In various embodiments of the above aspects or any other aspect of the present invention, the modified NLS comprises six copies of a NLS.

In certain embodiments, the modified crRNA comprises from 3′ to 5′ a first direct repeat (DR) sequence, a gRNA, and a second direct repeat sequence.

In certain embodiments, the crRNA comprises from 3′ to 5′ a first direct repeat sequence, a first gRNA, a second direct repeat sequence, a second gRNA, and a third direct repeat sequence.

In certain embodiments, any one of the direct repeats is about 19 nucleotides in length.

In certain embodiments, the screening identifies epigenetic interactions in the cancer cell.

In certain embodiments, the epigenetic interactions are synthetic sick/lethal interactions.

In certain embodiments, further comprising a crRNA library, wherein the crRNA library comprises a plurality of crRNAs targeting a plurality of genomic regions involved in epigenetic regulation.

In certain embodiments, each crRNA in the crRNA library comprises from 3′ to 5′ a first direct repeat sequence, a first gRNA, and a second direct repeat sequence.

In certain embodiments, each crRNA in the crRNA library comprises from 3′ to 5′ a first direct repeat sequence, a first gRNA, a second direct repeat sequence, a second gRNA, and a third direct repeat sequence.

In another aspect, the invention includes a composition comprising a first nucleic acid comprising a nucleotide sequence encoding an AsCas12a comprising a E174R mutation and a S542R mutation, and six nuclear localization sequences, and a second nucleic acid comprising a crRNA comprising a first direct repeat sequence, a first gRNA, and a second direct repeat sequence.

In another aspect, the invention includes a composition comprising a first nucleic acid comprising a nucleotide sequence encoding an AsCas12a comprising a E174R mutation and a S542R mutation, and six nuclear localization sequences, and a second nucleic acid comprising a crRNA comprising a first direct repeat sequence, a first gRNA, a second direct repeat sequence, a second gRNA, and a third direct repeat sequence.

In another aspect, the invention includes a composition comprising a first nucleic acid comprising a nucleotide sequence encoding an AsCas12a comprising a E174R mutation and a S542R mutation, and six nuclear localization sequences, and a crRNA library, wherein each crRNA in the crRNA library comprises from 3′ to 5′ a first direct repeat sequence, a first gRNA, a second direct repeat sequence, a second gRNA, and a third direct repeat sequence.

In another aspect, the invention includes a kit useful for combinatorial genetic screening comprising any one of the compositions of claims 30-32.

In another aspect, the invention includes a method of treating cancer in a subject in need thereof, the method comprising administering to the subject an inhibitor of Brd9 and an inhibitor of Jmjd6.

In another aspect, the invention includes a method of treating cancer in a subject in need thereof, the method comprising administering to the subject an inhibitor Jmjd6 and an inhibitor of Kat6a.

In various embodiments of the above aspects or any other aspect of the present invention, the cancer is leukemia.

In various embodiments of the above aspects or any other aspect of the present invention, the inhibitor is selected from the group consisting of a small molecule, an antibody, a CRISPR system, a miRNA, a drug, an inhibitory RNA, or a genome editing tool.

### BRIEF DESCRIPTION OF THE DRAWINGS

The foregoing and other features and advantages of the present invention will be more fully understood from the following detailed description of illustrative embodiments taken in conjunction with the accompanying drawings.

FIGS. 1A-1E depict optimization of an AsCas12a system to improve knockout efficiency in mammalian cells. FIG. 1A Configuration of the optimized vectors used for lentiviral AsCas12a and CRISPR RNA (crRNA) transduction experiments in mammalian cells. A Puromycin (Puro) resistance gene was used for selecting AsCas12a-positive cells, and a GFP reporter was linked with crRNA expression for tracking the crRNA-positive population in cellular competition assays. FIG. 1B. Experimental workflow of cellular competition assay to evaluate AsCas12a knockout efficiency. K562 cells were stably transduced with indicated AsCas12a vectors, followed by infection with crRNA virus. Flow cytometry-based tracking of crRNA-positive population over a period of time was used to calculate the negative selection phenotype of an indicated crRNA. FIGS. 1C-1E: Cellular competition assay to compare the knockout efficiency of the AsCas12a system with a variable number of NLS sequences (FIG. 1C), with or without an additional direct repeat (DR) 3′ of the crRNA (FIG. 1D), and with an AsCas12a variant (E174R and S542R) (FIG. 1E). Plotted are the crRNA-positive cells (normalized to the day 3 measurement) at the indicated timepoints during culturing. Two crRNAs were designed targeting PCNA, an essential gene for DNA replication. ‘e’ represents the exon. (n=3). All error bars shown represent s.d.

FIGS. 2A-2C depict a comparison of enAsCas12a and SpCas9 in single-gene knockout genetic screen. FIG. 2A: Experimental schematic of pooled dropout screen. A murine Mll-Af9/Nras^{G12D} acute myeloid leukemia cell line, RN2, was stably transduced with either enAsCas12a or SpCas9. A customized enAsCas12a crRNA library against 155 protein domains involved in epigenetic regulation was constructed. The library contains 3-5 crRNAs per domain. crRNAs were designed to target protein domains to maximize the frequency of generating functional knockout cells, yielding 787 crRNAs in total including positive and negative controls. Pooled enAsCas12a crRNA or SpCas9 sgRNA libraries targeting epigenetic regulatory domains were introduced into indicated RN2 cells via lentiviral transduction at low multiplicity of infection to ensure single copy viral integration. Genomic DNA from the initial time point, 2 days post-infection, and the finial time point, 12 days post-infection, were isolated for CRISPR RNA (either crRNA or sgRNA) cassette quantification by deep sequencing. A protein domain CRISPR Score (CS) was calculated by averaging the log² fold-change of all CRISPR RNA targeting a given protein domain. Fold-change=(final CRISPR RNA abundance+1)/(initial CRISPR RNA abundance). FIG. 2B: Result of pooled enAsCas12a dropout screen to identify epigenetic dependencies in RN2c12 cells. Scatter plot that compares the CSs of two independent replicates (r=0.96, Pearson correlation). FIG. 2C: Comparison of enAsCas12 and SpCas9 in pooled dropout screen against epigenetic regulators. Scatter plot that compares the CSs from enAsCas12a and SpCas9 screens in RN2 cells. Plotted is the average of two biological replicates. (r=0.93, Pearson correlation). (FIGS. 2B-2C) Red dots label known cancer drug targets in this Mll-A19 leukemia model.

FIGS. 3A-3F depict a enAsCas12-based combinatorial knockout screen in Mll-A19 leukemia identifying synthetic sick/lethal interactions of epigenetic regulators. (FIG. 3A) Schematic comparison of CRISPR RNA transcript expression vector architecture for enAsCas12a- and SpCas9-based double gene knockout. The enAsCas12a system requires a short DR sequence of 19-20 nucleotides (nt) for double gene knockout in comparison to the ˜352nt of an SpCas9 system. (FIG. 3B) Evaluation of the recombination frequency of dual-crRNAs in enAsCas12a system. Experimental workflow to quantify the uncoupling frequency of dual-crRNAs by deep sequencing. (FIG. 3C) An enAsCas12a-based double knockout screen reveals synthetic sick/lethal epigenetic regulator pairs in RN2c12 cells. Scatter plot that compares the expected and observed dual-crRNA CSs. The expected dual-crRNA CS was calculated based on a Gaussian distribution model of the experimental crRNAs in all possible combinations with negative control crRNAs. The observed dual-crRNA CS was calculated based on a Gaussian distribution model of all crRNA combinations of the two experimental protein domains. Blue dots represent the [negative crRNA]-[negative crRNA] control pairs and red dots represent potential synthetic sick/lethal genetic interactions (log² differential>2.5, qval<0.05). (FIG. 3D) Cellular competition assay in RN2c12 cells to validate the screen-identified potential synergistic interacting dual-crRNAs. Plotted are the dual-crRNA positive cells (normalized to the day 2 measurement) at the indicated timepoints during culturing. Each data point is comprised of pairwise combinations of the two indicated crRNAs. (n=2-3). (FIG. 3E) Viability of RN2c12 cells transduced with either Jmjd6 or Rosa26 crRNA and exposed to increasing concentrations of either dBrd9 or WM1119 for 5 days. dBrd9 is a selective BRD9 degrader and WM1119 is a KAT6A/B inhibitor. (n=3-5). (FIG. 3F) Venn diagram of RNA-seq data depicting the overlap of significantly up-regulated and down-regulated genes 236 upon double knockout with indicated dual-crRNAs in RN2c12 cells. All error bars shown represent s.d.

FIG. 4 depicts a schematic illustrating how a CRISPR-Cas12a processes a single transcript using its intrinsic RNase activity for multigene editing.

FIG. 5 illustrates that AsCas12a contains two potential nuclear export signal (NES) sequences. Multiple sequence alignment of the amino acid sequences of AsCas12a, MbCas12a, LbCas12a, and FnCas12a. As, Acidaminococcus sp; Mb, Moraxella bovoculi; Lb, Lachnospiraceae bacterium; Fn, Francisella novicida. The multiple sequence alignment was performed using Clustal Omega (https://www.ebi.ac.uk/Tools/msa/clustalo/). Conserved residues are labeled with a red background and white font. Residues with chemically similar R-groups are indicated with an outline and red font. The conserved catalytic RuvC-II domain is highlighted with a grey box. Predicted NES sequences using the NetNES website tool (http://www.cbs.dtu.dk/)1 are highlighted in blue. The canonical NES signal is LxxxLxxLxL. While AsCas12a contains 2 potential NES sequences, there are no predicted NES sequences in SpCas9.

FIGS. 6A-6C depict appending NLS and DR sequences improving AsCas12a knockout efficiency in mammalian cells. (FIG. 6A) Western blotting of AsCas12a, Lamin-B1, and α-Tubulin levels in both nuclear fraction and whole-cell lysates prepared from K562c12 cells. K562 cells transduced with the indicated AsCas12a constructs containing a variable number of NLS repeats. A representative experiment of two independent replicates is shown. (FIGS. 6B, 6C) Cellular competition assay to characterize the knockout efficiency of the AsCas12a system with variable numbers of NLS (FIG. 6B) and with or without an additional DR 3′ of the crRNA (FIG. 6C). Plotted are the crRNA+cell populations (normalized to the day 3 measurement) at the indicated timepoints. Three crRNAs were designed targeting the kinase domain of CDK1, an essential gene for cell cycle regulation. ‘e’ represents the exon. (n=3). All error bars shown represent s.d.

FIG. 7 depicts the observation that knockout by enAsCas12a with negative control crRNAs does not significantly affect cellular proliferation in K562 cells. Cellular competition assay of K562c12 cells transduced with indicated crRNAs. Plotted are the crRNA+cell populations (normalized to the day 3 measurement) at indicated timepoints. crNeg1-6 are non-targeting crRNAs. Additional crRNAs were designed to target intronic regions of the essential genes PCNA and CDK1. ‘i’ represents the intron. (n=3). All error bars shown represent s.d.

FIG. 8 depicts enAsCas12a showing robust knockout efficiency in RN2 cells. Cellular competition assay of RN2c12 cells transduced with indicated crRNAs. Plotted are the crRNA+cell populations (normalized to the day 2 measurement) at indicated timepoints. crRNAs targeting the Rosa26 and Ano9 loci serve as negative controls. crRNAs targeting Brd4, a known Mll-A19 leukemia dependency, and Rpa3, an essential gene for cell replication, serve as positive controls. ‘e’ represents the exon. (n=3). All error bars shown represent s.d.

FIG. 9 demonstrates that enAsCas12a performs as well as SpCas9 in a pooled library dropout screen against epigenetic regulators. Log 2 fold-change of cells containing specific CRISPR RNAs from enAsCas12a- and SpCas9-based dropout screens in RN2 cells. The fold-change of a given CRISPR RNA is capped at a maximum of 100. Bold lines represent the mean log 2 fold-change of all crRNAs targeting the indicated gene or protein domain. Plotted dots include all data points from two independent biological replicates.

FIG. 10 illustrates that a dual-crRNA expression vector of enAsCas12a shows robust knockout efficiency in RN2c12 cells. Cellular competition assay of RN2c12 cells transduced with indicated dual-crRNAs. Plotted are the dual-crRNA+cell populations (normalized to the day 2 measurement) at the indicated timepoints. crRNAs targeting the Rosa26 and Ano9 loci serve as negative controls. crRNAs targeting Brd4, a known MLL-AF9 leukemia dependency, and Rpa3, an essential gene for cell replication, serve as positive controls. ‘e’ represents the exon. (n=3). All error bars shown represent s.d.

FIG. 11 illustrates a cloning strategy to construct pairwise combinatorial AsCas12a dual-crRNA library. Experimental workflow to construct a customized dual-crRNA library. 21 domains of epigenetic regulators with moderate to no proliferative phenotype in RN2 cells were selected from the single-gene knockout screens performed in FIG. 2.

FIG. 12 illustrates the position effect in the enAsCas12a dual-crRNA expression vector in pooled screening. Cumulative curve that describes the percentage of dual-crRNAs with a preferential proliferation effect from a crRNA in either the first or second position of a dual-crRNA array. Positional difference on the x-axis is computed by subtracting the effect of a crRNA when paired with negative controls in the first position vs. when paired with negative controls in the second position. Negative values indicate a crRNA with a proliferation bias in the first position and positive values indicate a guide bias in the second position. The y-axis shows the percentage of dual-crRNAs that exceed the indicated positional difference.

FIG. 13 illustrates that Brd9&Jmjd6 and Kat6a&Jmjd6 synthetic sick/lethal interactions from pooled screening are validated in RN2c12 cells. Cellular competition assay of individual dual-crRNAs performed in RN2c12 cells. Plotted are the dual-crRNA positive cell populations (normalized to the day 2 measurement) at the indicated timepoints. Each data point is comprised of pairwise combinations of the two indicated crRNAs. Pairwise combinations include both rearrangements of an experimental gene pair. (n=2-3).

FIG. 14 illustrates that synthetic sick/lethal interactions introduced by dual-crRNAs cause a decrease in leukemia stem cell signature and an increase in myeloid differentiation signature. RNA-seq heatmap of gene expression changes following transduction with indicated dual-crRNAs in RN2c12 cells. Genes related to leukemia stem cell signature and myeloid differentiation programs are plotted, and ranked based on fold change in expression. Results are the average of 2-6 independent dual-crRNAs.

FIGS. 15A-15D illustrate optimization of Cas12a system in mammalian cells. (FIG. 15A) Quantification of GFP gene editing of indicated Cas proteins in a HEK293T GFP report cell line. (FIG. 15B) Cas12a can process dual-crRNAs and produce negative selection phenotype when targeting the Rpa3 essential gene in RN2 cells. (FIG. 15C) Comparison of Cas9 vs Cas12a systems in pooled genetic screens at the single gene level. Negative selection genetic screening against epigenetic regulators in RN2 cells. Well known AML epigenetic dependencies are labeled in red. (FIG. 15D) Evaluation of the dual crRNA shuffling frequency in Cas12a system.

FIGS. 16A-16C illustrate Cas12a-based combinatorial genetic screening. (FIG. 16A) Double knockout Cas12a screening reveals synergistic interacting epigenetic regulator pairs for proliferation of the AML line, RN2. (FIGS. 16B, 16C) Cellular competition assays to validate the synergistic interaction of Brd9 and Jmjd6 in RN2 cells using combinations of genetic knockout and chemical inhibitor approaches.

FIG. 17 is a schematic detailing the experimental workflow of double knockout CRISPR-Cas12a drop-out genetic screening to reveal HCC-specific epigenetic dependencies. A pooled CRISPR-Cas12a library with pairwise combinations of CRISPR RNA (crRNA) targeting epigenetic regulators will be introduced into Cas12a+ cancer cell lines. The library-transduced cells will be subjected to various screening conditions as indicated. Genomic DNA of indicated cell populations will be isolated and harvested for comparison of crRNA pair abundance via deep sequencing. The bioinformatic analysis will be performed to identify the crRNA pair showing a synergistic effect in inhibiting cell proliferation. GEM is genetically engineered tumor model; ICB is immune checkpoint blockade.

FIG. 18 illustrates a comparison of CRISPR Cas9 and Cas12a system. Schematic of a single transcript architecture of CRISPR RNA of Cas9 and Cas12a for single and double gene editing. The Cas12a system only requires a short stretch of ˜19 nucleotides (nt) for double gene editing.

FIGS. 19A-19D illustrate optimization of Cas12a system in mammalian cells. (FIG. 19A) Quantification of GFP gene editing of indicated Cas proteins in a HEK293T GFP report cell line. (FIG. 19B) Cas12a can process dual-crRNAs and produce negative selection phenotype when targeting the Rpa3 essential gene in RN2 cells. (FIG. 19C) Comparison of Cas9 vs Cas12a systems in pooled genetic screens at the single gene level. Negative selection genetic screening against epigenetic regulators in RN2 cells. Well known AML epigenetic dependencies are labeled in red. (FIG. 19D) Evaluation of the dual crRNA shuffling frequency in Cas12a system.

FIGS. 20A-20C illustrate Cas12a-based combinatorial genetic screening. (FIG. 20A) Double knockout Cas12a screening reveals synergistic interacting epigenetic regulator pairs for proliferation of the AML line, RN2. (FIGS. 20B, 20C) Cellular competition assays to validate the synergistic interaction of Brd9 and Jmjd6 in RN2 cells using combinations of genetic knockout and chemical inhibitor approaches.

FIGS. 21A-21B illustrate In vivo CRISPR Cas9 screening to identify combinatorial epigenetic regulators that mediate the response of immunotherapy. (FIG. 21A) Depletion scores under the indicted condition of in vivo and in vitro Cas9 screens in B16F10 tumors with a custom epigenetic library. (FIG. 21B) Survival curve of B16F10 tumor-bearing mice with the indicted gene knockout (Rosa26 or Setd1b) and treatment. aPD1, anti-PD1; aNK1.1, NK1.1 cell depleting antibody; aCD8, CD8 T cell depleting antibody.

### DETAILED DESCRIPTION

### Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends.

The term “CRISPR/Cas” or “clustered regularly interspaced short palindromic repeats” or “CRISPR” refers to DNA loci containing short repetitions of base sequences followed by short segments of spacer DNA from previous exposures to a virus or plasmid. Bacteria and archaea have evolved adaptive immune defenses termed CRISPR/CRISPR-associated (Cas) systems that use short RNA to direct degradation of foreign nucleic acids. In bacteria, the CRISPR system provides acquired immunity against invading foreign DNA via RNA-guided DNA cleavage.

The “CRISPR/Cas9” system or “CRISPR/Cas9-mediated gene editing” refers to a type II CRISPR/Cas system that has been modified for genome editing/engineering. It is typically comprised of a “guide” RNA (gRNA) and a non-specific CRISPR-associated endonuclease (Cas9). “Guide RNA (gRNA)” is used interchangeably herein with “short guide RNA (sgRNA)” or “single guide RNA (sgRNA). The sgRNA is a short synthetic RNA composed of a “scaffold” sequence necessary for Cas9-binding and a user-defined ˜20 nucleotide “spacer” or “targeting” sequence which defines the genomic target to be modified. The genomic target of Cas9 can be changed by changing the targeting sequence present in the sgRNA.

The “CRISPR-Cas12a” system refers to a type II CRISPR/cas gene editing system utilizing a Cas12a (also called Cpf1) nuclease. Cas12a offers several advantages over earlier developed Cas9-based system primarily associated with its endogenous pre-crRNA processing ability that does not require an additional tracrRNA, thus simplifying the process of multiplex gene targeting.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MEW class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Insertion/deletion”, commonly abbreviated “indel,” is a type of genetic polymorphism in which a specific nucleotide sequence is present (insertion) or absent (deletion) in a genome.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockin” as used herein refers to an exogenous nucleic acid sequence that has been inserted into a target sequence (e.g., endogenous gene locus). some embodiments, where the target sequence is a gene, a knockin is generated resulting in the exogenous nucleic acid sequence being in operable linkage with any upstream and/or downstream regulatory elements controlling expression of the target gene. In some embodiments, the knockin is generated resulting in the exogenous nucleic acid sequence not being in operable linkage with any upstream and/or downstream regulatory elements controlling expression of the target gene.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver. A transplant can also refer to any material that is to be administered to a host. For example, a transplant can refer to a nucleic acid or a protein.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based on the unexpected finding that CRISPR-Cas12a gene editing systems can be modified through a combination of mutations which enhance its DNA binding ability, modified crRNAs which have improved stability, and additional nuclear localization sequences (NLS) to improve editing efficiency. Also provided are compositions comprising the modified CRISPR-Cas12a system of the present invention. The invention also includes methods comprising the modified CRISPR-Cas12a system of the invention for screening for synergistic combinations of drug targets, as well as the treatment of cancer in subjects in need thereof.

### CRISPR-Based Gene Editing

CRISPR-Cas gene editing systems are classified into two classes (1 and 2) that are subdivided into six types (I through VI). Class 1 (types I, III and IV) systems rely on multiple Cas proteins to form a ribonucleoprotein complex called Cascade (CRISPR-associated complex for antiviral defense). Though accounting for about 90% of naturally-occurring CRISPR systems in archaea and bacteria, the complexity of Class 1 systems have made them undesirable for adapting into genome-editing systems. Class 2 systems, on the other hand, (types II, V and VI) use a single Cas protein. Class 2 CRISPR-Cas systems are naturally found almost exclusively in bacteria, and assemble a ribonucleoprotein complex, consisting of a CRISPR RNA (crRNA) and a Cas protein. The crRNA comprises sequences complementary to specific DNA sequences and acts to precisely guide the Cas nuclease to the target genomic sequined and short, adjacent genomic sequence called a protospacer adjacent motif (PAM) sequence. Cas protein complexes are adapted for precise genome editing by providing a crRNA with a designed guide sequence which is complementary to the sequence of the targeted DNA. The most widely characterized CRISPR-Cas system is the type II subtype II-A that is found in Streptococcus pyogenes (Sp), which uses the protein SpCas9, also called Cas9. Cas9 was the first Cas-protein engineered for use in gene editing. Class 2 type V CRISPR systems are further classified into 4 subtypes (V-A, V-B, V-C, V-U). V-A encodes the protein Cas12a (also known as Cpf1).

### CRISPR-Cas12a

As described herein, the discovery and characterization of the Class 2, type V CRISPR system, Cas12a/Cpf1 has enabled rapid genome editing of multiple loci in the same cell. Cas12a is a single component RNA-guided nuclease that can mediate target cleavage with a single crRNA, that is typically 42-44 nt in length, with the first 19/20 nt corresponding to the repeat sequence and the remaining 23-25 nt to the spacer sequence. While in type II CRISPR systems, such as Cas9-based systems, the maturation of crRNA requires host RNase III along with a trans-activating crRNA (tracrRNA), which is base paired with the pre-crRNA. The Cas12a protein, however, has been shown to process its own pre-crRNA into mature crRNAs, without the requirement of a tracrRNA, making it a unique effector protein with both endoribonuclease and endonuclease activities. After the pre-crRNA has been transcribed during the expression stage, it is cleaved by the Cas12a protein 4 nucleotides upstream of the hairpin structures formed by the CRISPR repeats, producing intermediate crRNA molecules which undergo further processing into mature crRNAs.

The Cas12a enzyme can be derived from any genera of microbes, including but not limited to, Parcubacteria, Lachnospiraceae, Butyrivibrio, Peregrinibacteria, Acidaminococcus, Porphyromonas, Lachnospiraceae, Porphromonas, Prevotella, Moraxela, Smithella, Leptospira, Lachnospiraceae, Francisella, Candidatus, and Eubacterium. In certain embodiments, Cas12a is derived from a species from the Acidaminococcus genus (AsCpf1).

The Cas12a protein adopts a bilobed structure formed by the REC and Nuc lobes. The REC lobe is comprised of REC1 and REC2 domains, and the Nuc lobe is comprised of the RuvC, the PAM-interacting (PI) and the WED domains, and additionally, the bridge helix (BH). The RuvC endonuclease domain of this effector protein is made up of three discontinuous parts (RuvC I-III). The RNase site for processing its own crRNA is situated in the WED-III subdomain, and the DNase site is located in the interface between the RuvC and the Nuc domains. Only the 5′ repeat region of the crRNA is involved in the assembly of the Cas complex. The 19/20 nt repeat region forms a pseudoknot structure through intramolecular base pairing. The crRNA is stabilized through interactions with the WED, RuvC and REC2 domains of the endonuclease, as well as two hydrated Mg2+ ions. This binary interference complex is then responsible for recognizing and cleaving target DNA sequences.

Cas12a-based CRISPR systems offer several differences from more traditional Cas9-based editing systems. Cas9 requires two RNA molecules: tracrRNA and a crRNA, whereas Cas12a requires only a single RNA molecule, the crRNA. Cas9 possesses two nuclease sites HNH and RuvC domains, while Cas12a possesses only one nuclease site in the RuvC domain. Additionally, Cas12a also possesses an RNA processing site. Additionally, there are distinct differences in the mechanisms employed by the two proteins when it comes to RNA processing, PAM recognition, target DNA binding and eventually catalysis. The single crRNA molecule of Cas12a systems simplifies and enables the process of targeting multiple genomic loci simultaneously.

In the context of formation of a CRISPR complex, “target sequence” refers to a sequence to which a crRNA sequence is designed to have some complementarity, where hybridization between a target sequence and a crRNA sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides.

### Modified CRISPR-Cas12a Systems

In certain embodiments, the Cas12a enzyme used in the invention is modified through specific mutations such that the function and efficiency of the enzyme is improved. There improvements can include, but are not limited to, increased DNA binding affinity, targeting range, on-target activities, fidelity, and decreased non-specific cellular toxicity (that is, toxicity not directly related to the targeted gene sequences) and the ability to target more genomic loci simultaneously as compared to unmodified systems and other modified systems known to the art. Previous studies have identified four mutations, which result in greater DNA-binding ability of the Cas enzyme resulting in more efficient gene editing activities on site with canonical and non-canonical PAMs relative to wildtype Cas12a. These mutations include S170R, E174R, S542R, and K548R (see Kleinstiver, et al. (2019) Nat Biotechnol. March; 37(3): 276-282.). Cas12a enzymes can be engineered to comprise one or all of these mutations, and the skilled artisan would recognize which mutations or combinations of mutations would be most beneficial to a particular application. In certain embodiments of the current invention, the Cas12a protein comprises an E174R mutation. In certain embodiments, the Cas12a protein comprises an S542R mutation. In certain embodiments, the Cas12a protein comprises both E174R and S542R mutations.

In certain embodiments, the modifications to the CRISPR-Cas12a system of the invention comprises a modified nuclear localization signal (NLS). These signals perform a key function by directing the CRISPR-Cas complex into the nucleus, wherein it is able to interact with genomic DNA. Previous studies known in the art have identified that altering the NLS of the Cas12a enzyme, typically by altering the number and position of NLS sequences, improves genome editing efficiency. (Liu, et al. (2019) Nucleic Acids Res. May 7; 47(8): 4169-4180) In particular, these studies found that engineered variants of Cas12a comprising two different nuclear localization sequences (NLS) on the C terminus demonstrated increased editing efficiency in mammalian cells. In certain embodiments of the current invention, the modification of the Cas12a involves the addition of six additional NLS sequences, which results in improved efficiency over wildtype NLS and engineered two NLS CRISPR-systems. In certain embodiments, when the six-NLS modification is combined with other modifications of the invention, the result is an optimal gene editing function.

In certain embodiments, the modifications to the CRISPR-Cas12a system of the invention comprises a dual direct repeat (DR) guide RNA. In some embodiments, the pre-crRNAs comprise full-length direct repeat (full-DR-crRNA) sequences with specific stem-loop G-C base substitutions. These modifications result in increased editing efficiencies, including editing rate, as compared with the standard mature crRNA framework of unmodified CRISPR-Cas12a systems and have been noted in previously published studies (Liu, et al. (2019) Nucleic Acids Res. May 7; 47(8): 4169-4180). In certain embodiments, the modified crRNA comprises from 3′ to 5′ a first direct repeat (DR) sequence, a gRNA, and a second direct repeat sequence. In certain embodiments, the crRNA comprises from 3′ to 5′ a first direct repeat sequence, a first gRNA, a second direct repeat sequence, a second gRNA, and a third direct repeat sequence. In certain embodiments, any one of the direct repeats is about 19 nucleotides in length. The inclusion of two direct repeats, also called dual direct repeats, increases DNA editing efficiency as compared to single direct repeats or standard mature crRNA frameworks.

In certain embodiments, the modifications to the CRISPR-Cas12a system of the invention comprises a combination of Cas12a enzyme mutations, a modified dual direct repeat guide RNA, and a modified nuclear localization signal comprising six repeated NLS sequences. In certain embodiments the Cas12a enzyme mutations comprise E174R and S542R mutations. While other modified CRISPR-Cas12a systems are known in the art, (see DeWeirdt, et al. (2020) Nat Biotechnol. July 13.) none possess the combination of modifications of the current invention. In some embodiments of the current invention, the modifications result in less than 10% non-specific cellular toxicity when the system is used to target up to four genes simultaneously, and with an editing efficiency of 85%-98%. These improvements offer a considerable improvement on the CRISPR systems commonly used in the art, particularly the decreased cellular toxicity, which can be a limiting factor in the application of CRISPR systems.

### Compositions

Certain aspects of the current invention comprise compositions comprising nucleic acids comprising a CRISPR-Cas12a system.

In one aspect, the invention provides a composition comprising a first nucleic acid comprising a nucleotide sequence encoding an AsCas12a comprising a E174R mutation and a S542R mutation, and six nuclear localization sequences, and a second nucleic acid comprising a crRNA comprising a first direct repeat sequence, a first gRNA, and a second direct repeat sequence.

In one aspect, the invention provides composition comprising a first nucleic acid comprising a nucleotide sequence encoding an AsCas12a comprising a E174R mutation and a S542R mutation, and six nuclear localization sequences, and a second nucleic acid comprising a crRNA comprising a first direct repeat sequence, a first gRNA, a second direct repeat sequence, a second gRNA, and a third direct repeat sequence.

In one aspect, the invention provides a composition comprising a first nucleic acid comprising a nucleotide sequence encoding an AsCas12a comprising a E174R mutation and a S542R mutation, and six nuclear localization sequences, and a crRNA library, wherein each crRNA in the crRNA library comprises from 3′ to 5′ a first direct repeat sequence, a first gRNA, a second direct repeat sequence, a second gRNA, and a third direct repeat sequence.

### Methods of Treatment

The present invention includes methods for treating or preventing cancer in a subject comprising administering to the subject a therapeutically effective amount of any of the compositions disclosed herein. This composition can be utilized as a prophylactic treatment, a therapeutic treatment, a personalized, subject-specific treatment, and/or a method of turning a ‘cold’ tumor into a ‘hot’ tumor, thus making it more susceptible to immunotherapy.

One aspect of the invention includes a method of identifying a combination of drug targets wherein a therapeutically synergistic effect is elicited when the combination of targets is treated, the method comprising administering to a cell a composition comprising a CRISPR-Cas12 system, wherein the CRISPR-Cas12a system comprises an enhanced Cas12a variant, a modified nuclear localization signal (NLS), and a modified CRISPR RNA (crRNA), whereby the CRISPR-Cas12a system mutates multiple genomic regions simultaneously, thereby identifying the drug target combination.

One aspect of the invention includes a method of treating cancer in a subject in need thereof, the method comprising administering a CRISPR-Cas12a system to a cancer cell from the subject, wherein the CRISPR-Cas12a system comprises an enhanced Cas12a variant, a modified nuclear localization signal (NLS), and a modified CRISPR RNA (crRNA), whereby the CRISPR-Cas12a system mutates multiple genomic regions simultaneously, and combinatorial genetic screening is performed, determining a cancer treatment based on the screening results, and administering the cancer treatment to the subject based on results of said screening.

One aspect of the invention includes a method of treating cancer in a subject in need thereof comprising administering to the subject a therapeutically effective amount of a composition comprising a nucleic acid and/or vector comprising a nucleic acid encoding a CRISPR-Cas12a system, wherein the CRISPR-12a system inhibits expression of at least one endogenous gene, thus treating the cancer in the subject. In some embodiments, the at least one endogenous gene is identified by the combinatorial genetic screening method of the above aspects of the invention.

In one aspect, the invention provides a method of treating cancer in a subject in need thereof, the method comprising administering to the subject an inhibitor of Brd9 and an inhibitor of Jmjd6.

In one aspect, the invention provides a method of treating cancer in a subject in need thereof, the method comprising administering to the subject an inhibitor Jmjd6 and an inhibitor of Kat6a.

In certain embodiments, one or more vectors driving expression of one or more elements of a CRISPR system are introduced into a cell, such that expression of the elements of the CRISPR system direct formation of a CRISPR complex at one or more target sites. For example, a Cas12a enzyme, and a crRNA could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction.

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity, and nucleic acid binding activity. Additional domains that can form part of a fusion protein comprising a CRISPR enzyme are described in U.S. Patent Appl. Publ. No. US20110059502, which is incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In one non-limiting embodiment, a vector drives the expression of the CRISPR system. The art is replete with suitable vectors that are useful in the present invention. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present invention may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector can be provided to a cell in the form of a viral vector. Viral vector technology is well known in the art and is described, for example, in Sambrook et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus, and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

The cell or cells utilized in the invention can be from any source known to one of ordinary skill in the art. The cells of the invention may be autologous, allogeneic or xenogeneic with respect to the subject undergoing treatment. In some embodiments, the cell is from a cancer cell line. In some embodiments, the cell is from the subject. In some embodiments, the cell from the subject is a cancer cell. In further embodiments, the cancer cell is from a tumor. In some embodiments, the subject is a mammal. In further embodiments, the subject is a human.

The compositions of the present invention may be administered in a manner appropriate to the disease to be treated or prevented. The quantity and frequency of administration will be determined by such factors as the condition of the patient, and the type and severity of the patient's disease, although appropriate dosages may be determined by clinical trials. Compositions of the invention can be administered in dosages and routes and at times to be determined in appropriate pre-clinical and clinical experimentation and trials. Compositions comprising cells or nucleic acids may be administered multiple times at various dosages. Administration of the cells or vectors of the invention may be combined with other methods useful to treat the desired disease or condition as determined by those of skill in the art.

In one embodiment, administering the therapeutically effective amount of the composition comprises a one dose, a two dose, a three dose, a four dose, or a multi-dose treatment. The administration of the modified cells or vectors of the invention may be carried out in any convenient manner known to those of skill in the art. In one embodiment, the cells are administered intratumorally.

The current invention includes compositions and methods for treating cancer. Types of cancer that can be treated include, but are not limited to, Acute Lymphoblastic Leukemia (ALL), Acute Myeloid Leukemia (AML), Adrenocortical Carcinoma, AIDS-Related Cancers, Kaposi Sarcoma, AIDS-Related Lymphoma, Primary CNS Lymphoma, Anal Cancer, Appendix Cancer (Gastrointestinal Carcinoid Tumors), Astrocytomas, Atypical Teratoid/Rhabdoid Tumor, Brain Cancer, Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors, Burkitt Lymphoma, Non-Hodgkin Lymphoma, Carcinoid Tumors, Carcinoma of Unknown Primary, Cardiac (Heart) Tumors, Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma (Mycosis Fungoides and Sézary Syndrome), Ductal Carcinoma In Situ (DCIS), Endometrial Cancer, Ependymoma, Esophageal Cancer, Esthesioneuroblastoma, Ewing Sarcoma, Extracranial Germ Cell Tumor, Eye Cancer, Intraocular Melanoma, Fallopian Tube Cancer, Fibrous Histiocytoma of Bone, Osteosarcoma, Gallbladder Cancer, Gastric Cancer, Stomach Cancer, Gastrointestinal Carcinoid Tumor, Gastrointestinal Stromal Tumors (GIST), Central Nervous System Germ Cell Tumors, Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis (Langerhans Cell), Hodgkin Lymphoma, Hypopharyngeal Cancer, Intraocular Melanoma, Islet Cell Tumors, Pancreatic Neuroendocrine Tumors, Kidney Cancer, Renal Cell Cancer, Langerhans Cell Histiocytosis, Laryngeal Cancer, Leukemia, Lip and Oral Cavity Cancer, Liver Cancer, Lung Cancer (Non-Small Cell and Small Cell), Lymphoma, Male Breast Cancer, Malignant Fibrous Histiocytoma of Bone and Osteosarcoma, Melanoma, Intraocular (Eye) Melanoma, Merkel Cell Carcinoma (Skin Cancer), Malignant Mesothelioma, Metastatic Cancer, Metastatic Squamous Neck Cancer with Occult Primary, Midline Tract Carcinoma With NUT Gene Changes, Mouth Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Nasal Cavity and Paranasal Sinus Cancer, Nasopharyngeal Cancer, Neuroblastoma, Non-Small Cell Lung Cancer, Oral Cancer, and Oropharyngeal Cancer, Ovarian Cancer, Pancreatic Cancer, Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System (CNS) Lymphoma, Primary Peritoneal Cancer, Prostate Cancer, Rectal Cancer, Recurrent Cancer, Retinoblastoma, Rhabdomyosarcoma, Salivary Gland Cancer, Sarcoma, Vascular Tumors, Uterine Sarcoma, Sezary Syndrome (Lymphoma), Small Cell Lung Cancer, Small Intestine Cancer, Soft Tissue Sarcoma, Squamous Cell Carcinoma, Stomach (Gastric) Cancer, Throat Cancer, Thymoma, Thymic Carcinoma, Thyroid Cancer, Transitional Cell Cancer of the Renal Pelvis and Ureter, Carcinoma of Unknown Primary, Ureter and Renal Pelvis, Transitional Cell Cancer, Urethral Cancer, Uterine Cancer, Vaginal Cancer, Vulvar Cancer, Wilms Tumor, and combinations thereof.

### Introduction of Nucleic Acids

In certain embodiments an expression system is used for the introduction of nucleic acids encoding a CRISPR-Cas12a gene editing system into the cells of interest. Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods including electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al., Hum Gene Ther., 12(8):861-70 (2001).

Biological methods for introducing a polynucleotide of interest into a host cell include use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Moreover, the nucleic acids may be introduced by any means, such as transducing the cells, transfecting the cells, and electroporating the cells. One nucleic acid may be introduced by one method and another nucleic acid may be introduced into the cell by a different method.

It should be understood that the methods and compositions that would be useful in the present invention are not limited to the particular formulations set forth in the examples. The following examples are put forth so as to provide those of ordinary skill in the art with a complete disclosure and description, and are not intended to limit the scope of what the inventors regard as their invention.

### Sources of Cells

In certain embodiments of the invention, cells are obtained from a subject. Non-limiting examples of subjects include humans, dogs, cats, mice, rats, pigs and transgenic species thereof. Preferably, the subject is a human. Cells can be obtained from a number of sources, including peripheral blood mononuclear cells, bone marrow, lymph node tissue, spleen tissue, umbilical cord, cancer cells and tumors. In certain embodiments, any number of cell lines available in the art, may be used. In certain embodiments, cells can be obtained from a unit of blood collected from a subject using any number of techniques known to the skilled artisan, such as Ficoll separation. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

Cells can also be frozen. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some aspects, the invention includes a kit useful for combinatorial genetic screening comprising any one of the compositions of the invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, Molecular Cloning: A Laboratory Manual”, fourth edition (Sambrook et al. (2012) Molecular Cloning, Cold Spring Harbor Laboratory); “Oligonucleotide Synthesis” (Gait, M. J. (1984). Oligonucleotide synthesis. IRL press); “Culture of Animal Cells” (Freshney, R. (2010). Culture of animal cells. Cell Proliferation, 15(2.3), 1); “Methods in Enzymology” “Weir's Handbook of Experimental Immunology” (Wiley-Blackwell; 5 edition (Jan. 15, 1996); “Gene Transfer Vectors for Mammalian Cells” (Miller and Carlos, (1987) Cold Spring Harbor Laboratory, New York); “Short Protocols in Molecular Biology” (Ausubel et al., Current Protocols; 5 edition (Nov. 5, 2002)); “Polymerase Chain Reaction: Principles, Applications and Troubleshooting”, (Babar, M., VDM Verlag Dr. Müller (Aug. 17, 2011)); “Current Protocols in Immunology” (Coligan, John Wiley & Sons, Inc. Nov. 1, 2002).

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

The material and methods employed in these experiments are now described.

Cell lines: RN2, a murine Mll-Af9 cell line, was derived from an Mll-A19 knock-in mouse29. RN2, and its derivative cell lines, were cultured in RPMI-1640 supplemented with 10% FBS. HEK293T (ATCC Cat #CRL-3216, RRID:CVCL_0063), NIH3T3 (ATCC Cat #CRL-1658, RRID:CVCL_0594), B16-F10 (ATCC Cat #CRL-6475, RRID:CVCL_0159), and A549 (ATCC Cat #CCL-185, RRID:CVCL_0023) cells were cultured in DMEM supplemented with 10% bovine calf serum. K562 (ATCC Cat #CCL-243, RRID:CVCL_0004), MOLM13 (DSMZ Cat #ACC-554, RRID:CVCL_2119), and HEL (ATCC Cat #TIB-180, RRID:CVCL_2481) cells were cultured in RPMI-1640 supplemented with 10% bovine calf serum. All cell culture media was supplemented with 1% penicillin/streptomycin. All cell lines were cultured at 37° C. with 5% CO2 and were periodically tested to be mycoplasma-negative.

Vector construction and crRNA cloning. The protein-coding sequence of AsCas12a (RRID:Addgene_84739) was subcloned into a lentiviral expression vector, EFS-FLAG-P2A-Puro (RRID:Addgene_108100). NLS sequences were incorporated into overlapping DNA oligonucleotides homologous to the Cas-containing backbone by PCR. AsCas12a (E174R, S542R) was cloned in two rounds of mutagenesis PCR on the completed WT AsCas12a-6×NLS vector to generate a final lentiviral vector, opAsCas12a (pRG232, Addgene_149723). All vector cloning was performed using the In-Fusion HD Cloning system (TBUSA).

The AsCas12a crRNA expression vector (pRG212, Addgene_149722) was built by replacing the sgRNA region in the CROPseq-Guide-Puro plasmid (RRID:Addgene_86708) with AsCas12a DRs flanking a short filler that contained BsmbI restriction sites. In addition, the puromycin resistance cassette was replaced with EGFP-P2A-Neomycin, subcloned from LRG2.1-Neomycin (RRID:Addgene_125593).

The resulting pRG212 (EFS-GFP-P2A-Neo-U6-crRNA) vector was BsmbI-digested, and crRNAs were ligated in with T4 DNA ligase (NEB). Single-crRNA and dual-crRNA were cloned by annealing and phosphorylating complementary DNA oligonucleotides with T4 polynucleotide kinase (NEB).

Lentivirus transduction. Lentivirus was produced by transfecting HEK293T cells with helper plasmids VSVG and psPAX2 (RRID:Addgene_12260) using polyethylenimine (Polysciences, PEI 25000) in a mass ratio of 4:2:3 for plasmid DNA:VSVG:psPAX2. Media was replaced ˜6-8 h post transfection, and viral supernatant was collected several times within 24-72 h of transfection. Supernatant was passed through a 0.45 μm PVDF filter before use (Millipore). Lentivirus was added to target cell lines with 8 μg/mL Polybrene (Sigma #H9268) and centrifuged at 650×g for 25 min at room temperature. Media was changed 15 h post infection. Antibiotics (1 μg/mL puromycin and/or 50 mg/mL G418) were added 15 h post infection when selection was needed.

GFP disruption assay. HEK293T cells were first lentivirally transduced with a destabilized GFP (d2GFP) reporter (derived from Addgene #14760). The resulting HEK293T d2GFP reporter line was then transiently transfected with indicated AsCas12a vector and a vector expressing GFP-targeting crRNA. This d2GFP HEK293T reporter line was used to evaluate the knockout efficiency of seven Addgene-available Cas12a systems (Butyrivibrio sp., Thiomicrospira sp. XS5, Moraxella bovoculi, Prevotella bryantii, Bacteroidetes oral, Lachnospiraceae bacterium, and Acidaminococcus). In addition, AsCas12a vector contained 1, 2, 4, or 6 SV40 or c-Myc NLS cloned into either the N- or C-terminus. The crRNA vector contained an expression marker for mCherry for successful transfection tracking. Cells were cultured for 3 days and subjected to flow cytometry to measure knockdown of GFP using the Guava Easycyte 10 HT instrument (Millipore). Percent GFP disruption is plotted. Two independent GFP-targeting crRNA are presented.

Protein extraction and western blot. Approximately 5×106 K562-AsCas12a-xNLS cells were collected for nuclear extraction. After an initial PBS wash, nuclear protein fractions were isolated with the Compartment Protein Extraction Kit (Millipore, Catalog #2145) according to the manufacturer's instructions. To prepare whole cell lysates, 2×106 cells were resuspended in Laemmli sample buffer (Bio-Rad) containing 5% β-mercaptoethanol, and then denatured at 95° C. for 10 min. The protein extracts were separated on a 4-15% TGX gel (Bio-Rad), transferred to a nitrocellulose membrane, and analyzed by immunoblotting. All primary antibodies used at 1:1000 dilutions: FLAG (Sigma-Aldrich Cat #F1804, RRID:AB_262044), Lamin-B1 (Abcam Cat #ab16048, RRID:AB_10107828), and α-Tubulin (Sigma-Aldrich Cat #T6199, RRID:AB_477583). Secondary antibodies used: anti-Rabbit (LI-COR Biosciences Cat #925-32211, RRID:AB_2651127), anti-Mouse (Thermo Fisher Scientific Cat #A-21058, RRID:AB_2535724). Blots were imaged on a Licor Odyssey. Relative abundance of protein was quantified via gel densitometry with ImageJ software. AsCas12a protein levels in the nuclear fraction were first normalized to Lamin-B1 levels and then to the corresponding 1×NLS samples.

Competition-based cellular proliferation assays. Indicated AsCas12a-expressing cell lines were infected with a lentivirus (pRG212) encoding crRNA, targeting essential genes or known cancer dependencies, linked to a GFP reporter, to track successful transduction, at a multiplicity of infection (MOI) of around 0.2-0.5. The percentage of crRNA-positive (GFP-positive) cell population was monitored over time using the Guava Easycyte 10 HT instrument (Millipore). To assess the impact of a specific single-crRNA or dual-crRNA on cellular proliferation, final time point GFP % was divided by initial time point GFP % to calculate the negative selection fold-change of the crRNA-positive cell population.

Epigenetic regulator single-gene knockout screening. Overall, 23-nt crRNA sequences were designed in Benchling to target functional protein domains using the protospacer adjacent motif (PAM) 5′-TTTV. The AsCas12a library gene list was adapted from a previously published SpCas9 pooled genetic screening against epigenetic regulators. Approximately five crRNAs were designed for each domain, though some domains contain fewer (≥3 crRNAs) due to limited TTTV PAM abundance. An oligonucleotide pool containing the crRNA sequences flanked by BsmbI recognition sites and 24-nt primer sites was synthesized (Twist Bioscience). The pool was amplified with Phusion Hot Start Flex DNA Polymerase (NEB) over 20 cycles with an annealing temperature of 65° C. The PCR product was gel-purified (Macherey-Nagel) and ligated into the BsmbI-digested pRG212 backbone using Golden Gate cloning. The ligation product was electroporated into MegaX DH10B electrocompetent cells (Invitrogen) with an ECM 630 Electroporation System (BTX) according to the manufacturer's protocol. The cloned crRNA library was cultured overnight at 30° C. to minimize recombination, and the DNA was extracted with the PureLink HiPure Plasmid Maxiprep Kit (Invitrogen). Diversity of crRNA abundance was verified by Sanger sequencing randomly-picked bacterial colonies. To confirm that all designed crRNAs were cloned into the pRG212 vector, deep sequencing analysis was performed on either a MiSeq or NextSeq instrument (Illumina).

Single-gene knockout screening using either SpCas9, WT AsCas12a, or opAsCas12a was performed in murine Mll-Af9/NrasG12D acute myeloid leukemia cell line, RN2, stably expressing the corresponding Cas protein. The RN2c9 cell line was generated as a serial dilution-derived SpCas9 positive clonal line. Lentivirus containing the pooled library was prepared and used to infect cells as described above. The lentivirus was titrated to achieve an MOI of ˜0.3-0.5 to ensure single-copy viral integration, and cells were maintained at exponential growth. Cells were harvested on day 2 post infection as a reference for the fully represented pooled CRISPR RNA library. Cells were then cultured for 10 additional days and harvested on day 12 post infection. Either AsCas12a crRNA or SpCas9 sgRNA positive cells were cultured at ≥1000×CRISPR RNA coverage to maintain full representation of the pooled CRISPR RNA library. Harvested cells were washed in PBS, pelleted, and stored at −80° C. until genomic DNA extraction. Genomic DNA was isolated from the stated initial and final timepoints, and CRISPR RNA cassette quantification was conducted through deep sequencing.

Design principle single-gene knockout screening. Overall, 23-nt crRNA sequences were designed in Benchling to target the functional protein domains, nondomain coding regions, and noncoding regions of pan-essential and leukemia-essential genes using variable PAMs 5′-TTTV (TTTA, TTTC, or TTTG). On average, 40 crRNAs were designed per gene. Domain crRNAs target Pfam- or UniProt-identified domain regions of NCBI RefSeq exons, determined using the UCSC genome browser. Nondomain crRNAs target nondomain exonic regions of NCBI RefSeq genes. Noncoding crRNAs target intronic or untranslated regions of NCBI RefSeq genes. crRNAs with a perfect match to any off-target sites in the genome were filtered out. The final 2298 AsCas12a crRNA list for analysis is included (see Grier, et al. (2020) Nature Communications 11: 3455). An oligonucleotide pool containing crRNA sequences was ligated into the crRNA vector according to the aforementioned single-gene knockout screening protocol. To confirm that all designed crRNAs were cloned into the pRG212 vector, deep sequencing analysis was performed on a MiSeq instrument (Illumina).

Single-gene knockout screening using opAsCas12a was performed in RN2 cells stably expressing the Cas protein. Lentivirus containing the pooled library was prepared and used to infect cells as described above. Cells were harvested on day 2 post infection as a reference for the fully represented pooled CRISPR RNA library. Cells were then cultured for 6, 8, and 10 additional days and harvested. crRNA-positive cells were cultured at ≥×1000 crRNA coverage to maintain full representation of the pooled crRNA library. Harvested cells were washed in PBS, pelleted, and stored at −80° C. until genomic DNA extraction.

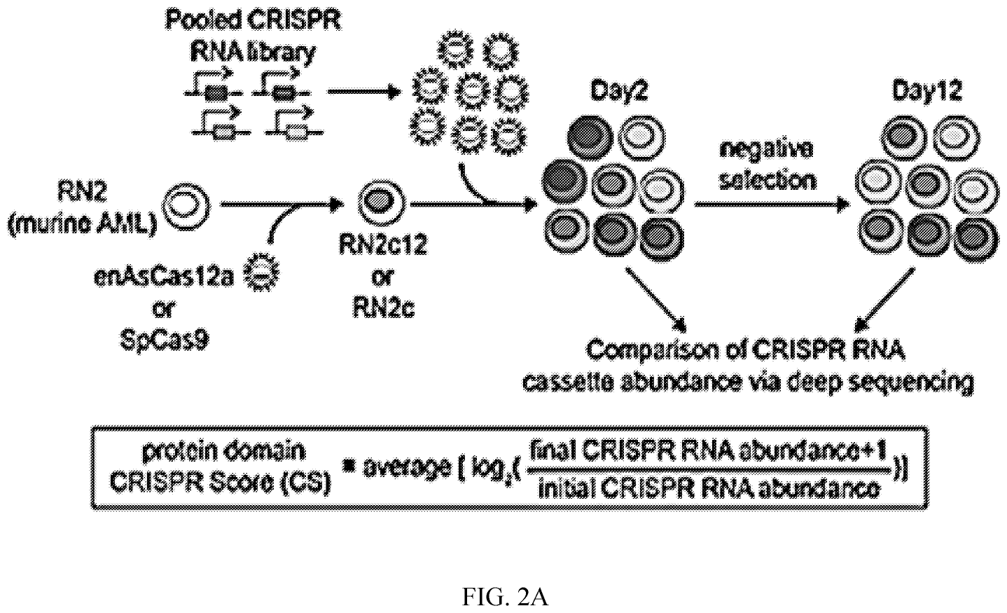

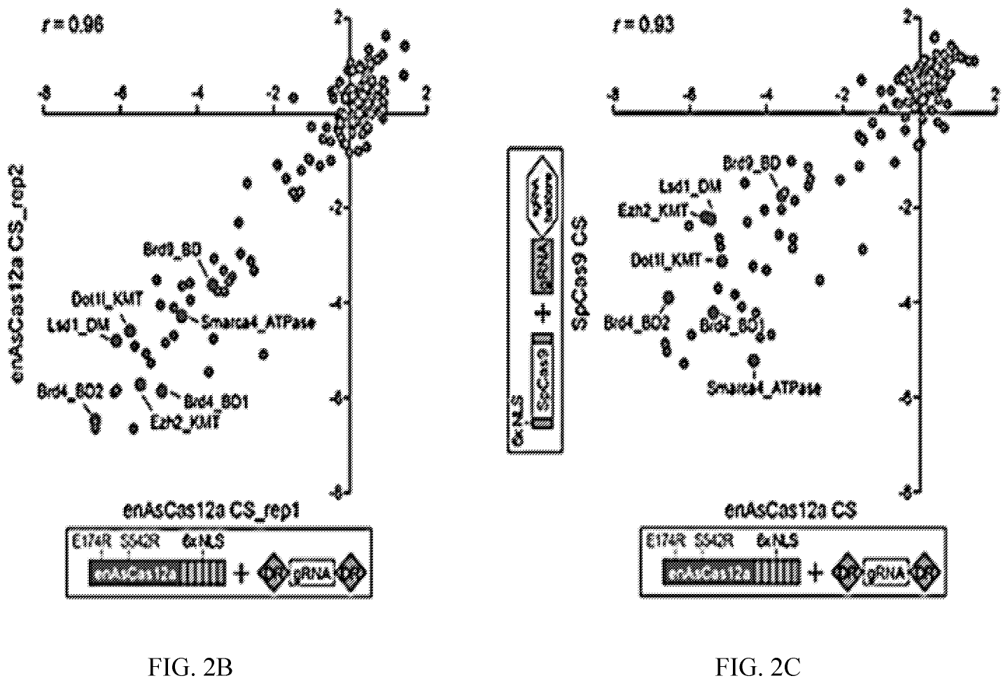

Double knockout screening. Twenty-one domains of epigenetic regulators with moderate-to-no proliferation phenotypic effect in RN2 cells were adapted from the single-gene knockout screens performed in FIG. 2. Three crRNAs from the single-gene knockout screening were selected for each gene. Separate oligonucleotide pairs were ordered for each crRNA for the first and second positions in the dual-crRNA expression vector. The oligonucleotide pairs were all phosphorylated and annealed separately, before being pooled together with the BsmbI-digested pRG212 backbone in a ligation reaction (T4 DNA ligase). The ligated product was electroporated into the MegaX DH10B cells. Lentivirus production of the pooled library, infection of the opAsCas12a-transduced RN2 cells, and the screening itself were carried out as in the single-gene knockout screening described above.

Deep sequencing library construction. Genomic DNA was isolated from the screening samples collected at the initial and final timepoints with the Quick-DNA Mini or Midiprep Plus kit (Zymo Research) following the manufacturer's protocol. An initial PCR step was performed to amplify the CRISPR RNA cassette and to incorporate unique different-length stacking barcodes to each sample. Multiple reactions with 200-400 ng DNA were run in parallel PCRs to ensure ˜1000× crRNA library representation. The Illumina sequencing adapters were then added during eight cycles of PCR. All primer sequences are listed in of (Gier, et al. (2020) Nature Communications 11: 3455). The final library product was quantified and qualified using the Bioanalyzer Agilent DNA 1000 kit (Agilent #5067-1504) and Qubit dsDNA High Sensitivity Kit (Invitrogen). Individual samples were pooled in equal molar ratio. The single-gene knockout screening libraries were pair-end sequenced on the MiSeq (Illumina) with MiSeq Reagent V3 150-cycle kit (Illumina), and the double knockout screening libraries were single-end sequenced on the NextSeq (Illumina) with NextSeq Mid-Output V2.5 150-cycle kit (Illumina).

Single-gene level knockout screening data analysis. Briefly, the sequence data were demultiplexed and trimmed to contain only the CRISPR RNA (either AsCas12a crRNA or SpCas9 sgRNA) sequence cassettes. The read count of each individual CRISPR RNA was calculated with no mismatches permitted in comparison to the reference CRISPR RNA sequences. Individual CRISPR RNAs with a read count lower than 50 in the initial time point were discarded. All samples were normalized to the same number of total reads. A protein domain CS was calculated by averaging the LFC of all CRISPR RNA targeting a given protein domain. LFC=(final CRISPR RNA abundance+1)/(initial CRISPR RNA abundance). The fold-change of a given CRISPR RNA was capped at a maximum of 100. This data analysis was performed similarly as described previously.

Double knockout screening data analysis. A BLAST reference index of all sequence combinations was assembled using makeblastdb (version 2.6.0). For each sample, adapter sequences were trimmed using CutAdapt (version 1.16), and then mapped to the BLAST index above using blastn. Blast queried sequence mapping was restricted with three rules: (1) having only one high-scoring dual-crRNA, (2) reporting only one aligned target (“−max_hsps 1−max_target_seqs 1”), and (3) allowing no more than two mismatching nucleotides (including mismatches, indels and unaligned tails). Read counts across two replicates were condensed for analysis. Dual-crRNA read counts were normalized to one million reads per library. A protein domain CS was calculated by averaging the LFC of all CRISPR RNA targeting a given protein domain. LFC=(final CRISPR RNA abundance+1)/(initial CRISPR RNA abundance). The fold-change of a given CRISPR RNA was capped at a maximum of 100.

The designed library contained both orientations of each dual-crRNA pair with an experimental crRNA in a fixed position joined to all 22 possible negative control crRNAs in the other position (n.b. exp1_neg1 is not grouped with neg1_exp1 in downstream analysis). Any experimental crRNA with less than four different negative controls in the other position detected on sequencing was eliminated from further analysis (e.g. if only three different negative control crRNAs in position two are detected paired with experimental crRNA “1” in the first position, then all experimental crRNA “1” pairings in either position are eliminated from the study). From the dual-crRNAs that met these criteria, any dual-crRNAs that had less than 8 detected dual-crRNA pairs were further eliminated. In the end, each gene in the analysis had at least two valid crRNAs designed for it.

To quantify the effect of each experimental crRNA, a Gaussian distribution was fitted using all the observed LFC values of the experimental crRNA in combination with all negative control crRNAs. The fitting was performed in a position sensitive way (i.e. fg (exp1_neg) is not the same as fg (neg_exp1)). To estimate the expected phenotypic effect of an experimental-experimental dual-crRNA pairing, the Gaussian distributions of each experimental crRNA paired with negative control crRNAs were multiplied. (i.e. fg (exp1_exp2)=fg (exp1_neg)×fg (neg_exp2) and fg (exp2_exp1)=fg (exp2_neg)×fg (neg_exp1)).

To estimate the expected phenotypic effect at the gene level, a Gaussian distribution model was built based on the combination of all the experimental crRNAs targeting a given gene and the negative control crRNAs. Similar to the analysis at the crRNA level, to estimate the expected phenotypic effect of two experimental genes, two Gaussian distributions of each gene were multiplied. To calculate the observed phenotypic effect of two genes, a Gaussian distribution was fitted by all the CSs of crRNA combinations targeting the two experimental genes. To identify potential synthetic sick/lethal genetic interactions, a Kolmogorov-Smirnov test was performed on the expected Gaussian distribution and the observed Gaussian distribution of two genes paired with an FDR cutoff of 0.05 and a LFC of 2.5.

Double knockout uncoupling data analysis. A BLAST reference index of all library dual-crRNA cassette sequences and all possible cassette recombination products was assembled using makeblastdb (version 2.6.0). For each sample, adapter sequences were trimmed using CutAdapt (version 1.16), reads shorter than 60 bp were discarded (empty crRNA vector), and filtered reads were mapped to the BLAST index above using blastn. Blast queried sequence mapping was restricted with three rules: (1) having only one high-scoring dual-crRNA, (2) reporting only one aligned target (“−max_hsps 1−max_target_seqs 1”), and (3) allowing no more than two mismatching nucleotides (including mismatches, indels and unaligned tails). For each sample, the number of reads mapped to each reference sequence was counted. Uncoupling frequency was calculated as the fraction of all reads mapped to a cassette recombination product divided by total reads mapped to any reference sequence.

Small molecule inhibitor treatment. Jmjd6-deficient cells were generated by infecting RN2c12 cells with a validated Jmjd6 crRNA. crRNA-positive (GFP positivity) cells were selected with G418 to achieve ≥95% purity and validated with TIDE analysis. A control cell line was also generated by transducing a Rosa26 crRNA in the same way. To test the cell growth of crJmjd6- and crRosa26-containing RN2c12 cells upon dBRD9 (provided by Jun Qi) or WM-1119 (Tocris, #6692) treatment, 1000 cells were plated into 48-well plates. Serially diluted concentrations of dBRD9, WM-1119, or 0.1% DMSO (negative control) was used for the treatment. After a 5-day incubation, cell viability was measured using CellTiter-Glo Luminescent Cell Viability Assay kit (Promega #G7570) with Synergy HTX microplate reader (Biotek). A 1:3 ratio of reagent to PBS was used, and all other steps were conducted according to the manufacturer's protocol.

RNA-seq and data analysis. RN2c12 cells were harvested on day 6 post infection with indicated crRNAs. To enrich the crRNA-positive population, G418 selection was applied on day 2 post infection. RNA was isolated from crRNA-transduced RN2c12 cells just before the onset of the growth arrest phenotype in order to capture the immediate transcriptional program changes. Total RNA was isolated using Direct-zol™ RNA Miniprep Plus kit (Zymo Research #R2072) following the manufacturer's protocol. The quality of isolated RNA was verified using the RNA 6000 Nano Bioanalyzer kit (Agilent #5067-1511). Only RNA with a RIN≥9 was used for subsequent library construction. RNA-seq libraries were prepared with the QuantSeq 3′ mRNA-Seq Library Prep Kit FWD for Illumina (Lexogen). 500 ng of total RNA was used for the initial input, and 12 cycles of PCR were used in barcode amplification. Quality of the RNA-seq libraries was assessed using the High Sensitivity DNA Bioanalyzer kit (Agilent #5067-4626). Libraries were pooled together and sequenced on the NextSeq 500/550 Platform with single-end reads of 75 bases (Illumina).

Sequencing reads were mapped to the reference mouse genome (mm10) using Lexogen Quantseq 2.3.1 FWD platform. Low quality reads, poly (A) read-through, and adapter contamination were removed with BBDuk, and raw reads were mapped to mm10 using STAR Aligner with modified ENCODE settings. HTSeq-count was used to generate gene read count files. Mapped raw reads were subjected to DESeq2 (1.14.1) to identify differentially expressed genes with default settings, and read counts >2 were considered expressed. Genes with |log 2FC|≥1 and p<0.05 were considered significantly and strongly up- or downregulated genes. To generate heatmaps, raw read counts were first converted to read per kilobase of transcript per million (RPKM) using the rpkm function in R (version 3.3.2). RPKM≥0.2 were considered expressed. The heatmap was generated using the heatmap.2 function in R, with RPKM in each condition averaged. Gene lists for the Leukemia Stem Cell and Myeloid Differentiation gene set enrichment analyses are provided in (Gier, et al. (2020) Nature Communications 11: 3455).

The results of the experiments are now described.

### Example 1: Optimization of a AsCas12a Knock-Out System

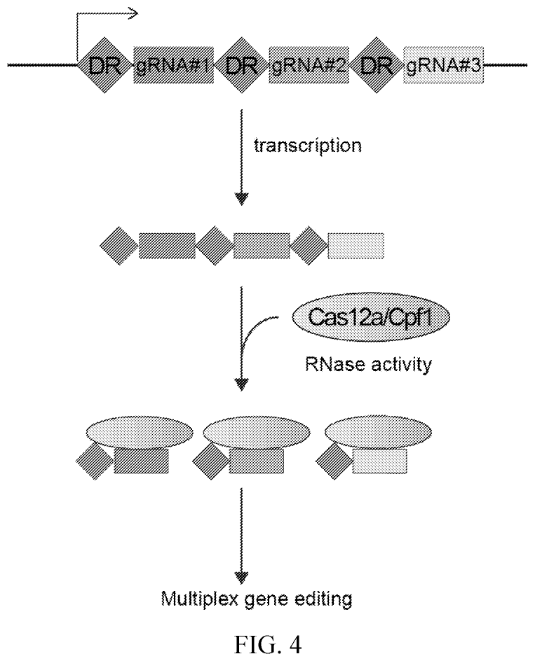

Combinatorial genetic screening, a high-throughput strategy to perturb multiple genes simultaneously, holds great promise for discovering therapeutically tractable synthetic sick/lethal interactions across diverse disease types, including cancers. Currently, Cas9-based methods are efficient in single-gene knockout screening but underperform in combinatorial genetic screening, primarily due to the high recombination frequency and complicated cloning of the dual CRISPR-Cas9 RNA (sgRNA) expression vector. Cas12a (formerly Cpf1), an RNA-programmable DNA endonuclease similar to Cas9, has the potential to overcome these limitations. Unlike Cas9, Cas12a has the intrinsic RNase activity to process its own crRNA array, enabling multi-gene editing from a single RNA transcript (FIG. 4). However, the low gene editing efficiency of Cas12a in mammalian cells restricts its application in combinatorial genetic screening, with no reported Cas12a-based combinatorial negative-selection ‘dropout’ screen.

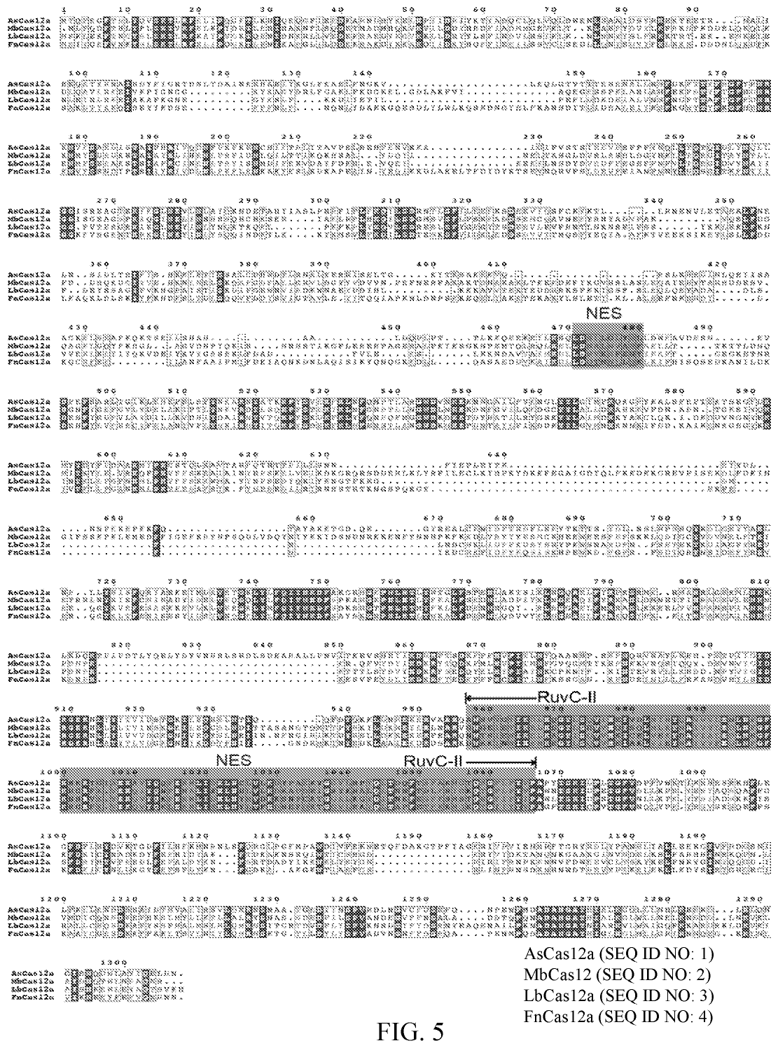

To optimize Cas12a activity, multiple Cas12a orthologs were surveyed for knockout efficiency. Seven Addgene-available Cas12a systems were transiently expressed in HEK293T cells to disrupt a GFP reporter in order to measure knockout efficiency. Consistent with published work, Acidaminococcus Cas12a (AsCas12a) demonstrated high gene editing activity. Nevertheless, the knockout efficiency of AsCas12a in human cells is approximately one third of that of Streptococcus pyogenes Cas9 (SpCas9), though AsCas12a is as biochemically active as SpCas9. Without wishing to be bound by theory, it was hypothesized that suboptimal nuclear localization might be partially responsible for low knockout efficiency of AsCas12a. Inspection of the amino acid sequence revealed that there are two potential nuclear export signals (NES) in AsCas12a, one of which is located in the highly conserved catalytic RuvC domain (FIG. 5).

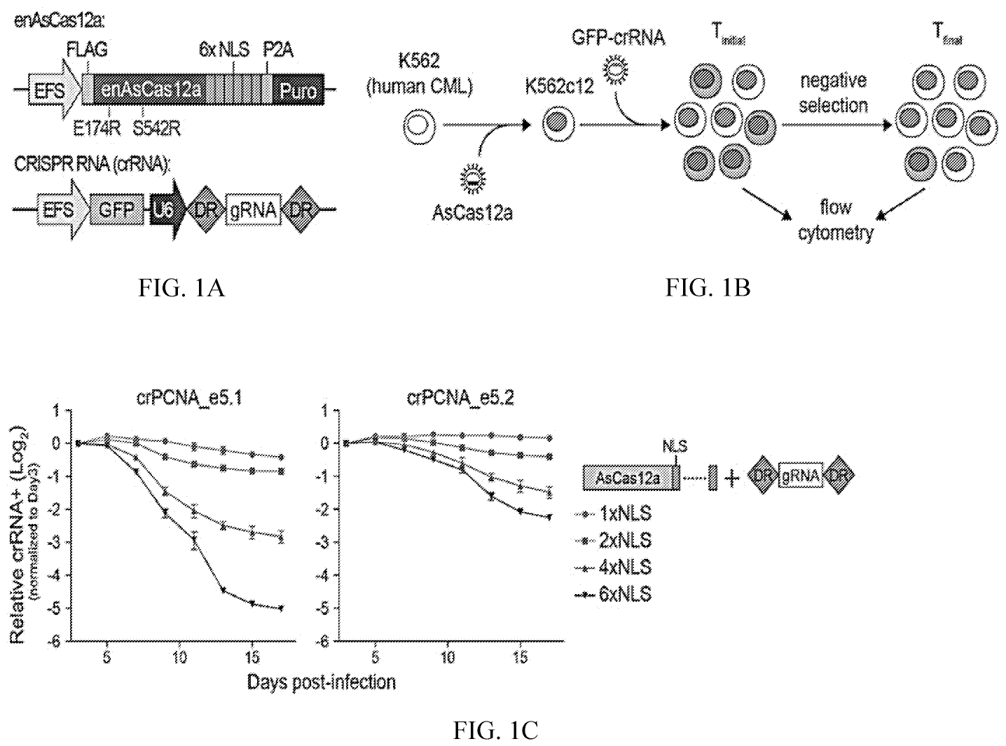

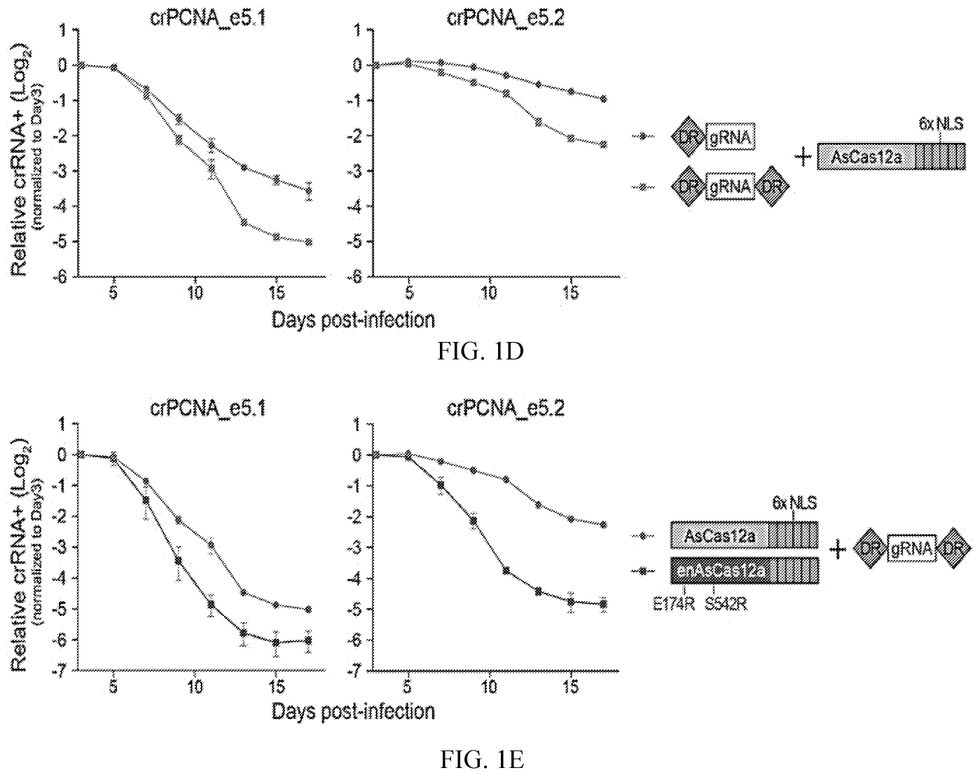

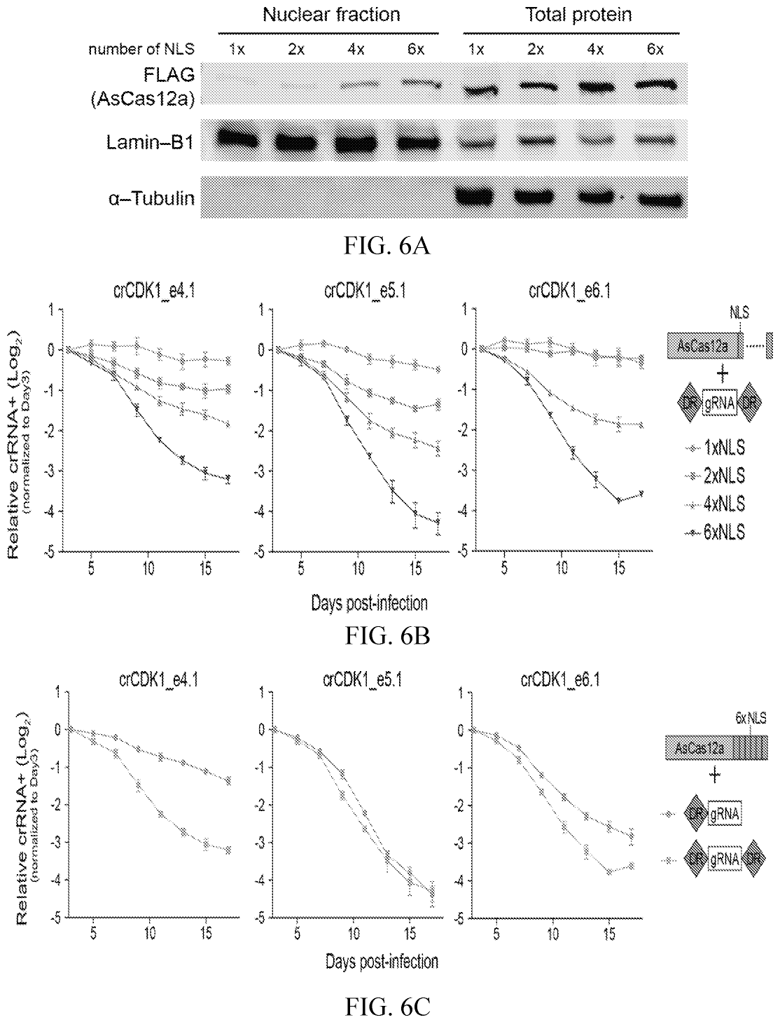

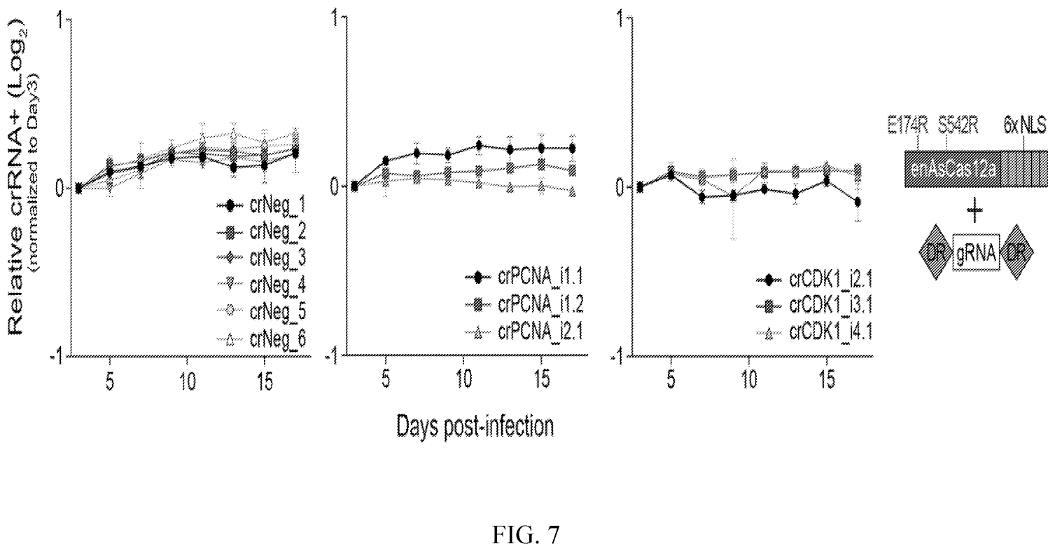

To address the low knockout efficiency in mammalian cells, the AsCas12a system was subjected to three levels of optimization. In order to evaluate the knockout efficiency of the system, AsCas12a and its crRNA were expressed using a two-vector lentiviral expression strategy and conducted a competition-based cellular proliferation assay, a reliable proxy for dropout screening (FIG. 1A, 1B). crRNAs targeting known essential genes were designed, including replication protein PCNA and cyclin-dependent kinase CDK1. AsCas12a knockout efficiency was then assessed by measuring the negative selection strength of essential gene-targeting crRNAs in a 16-day time-course. First, introducing six NLSs and adding a DR 3′ of the crRNA, which might protect it from 3′ exonuclease degradation, significantly increased AsCas12a knockout efficiency in K562 cells (FIG. 1C, 1D, and FIG. 6). Next, knockout efficiency was enhanced by introducing E174R and S542R mutations in AsCas12a that has increased DNA binding affinity and activity, termed enAsCas12a (FIG. 1E). EnAsCas12a with non-targeting crRNAs and crRNAs targeting introns of PCNA and CDK1 in K562 cells were then tested. Observations found no obvious effect on cellular proliferation, suggesting that the optimized enAsCas12a system was not toxic to the cells (FIG. 7). Together, these data establish that the combination of 6×NLS, dual-DR crRNA, and enAsCas12a protein enhances knockout efficiency (this optimized enAsCas12a system was used for all remaining experiments) (FIG. 1A).

### Example 2: Comparison of enAsCas12a and SpCas9 in a Single-Gene Knockout Genetic Screen

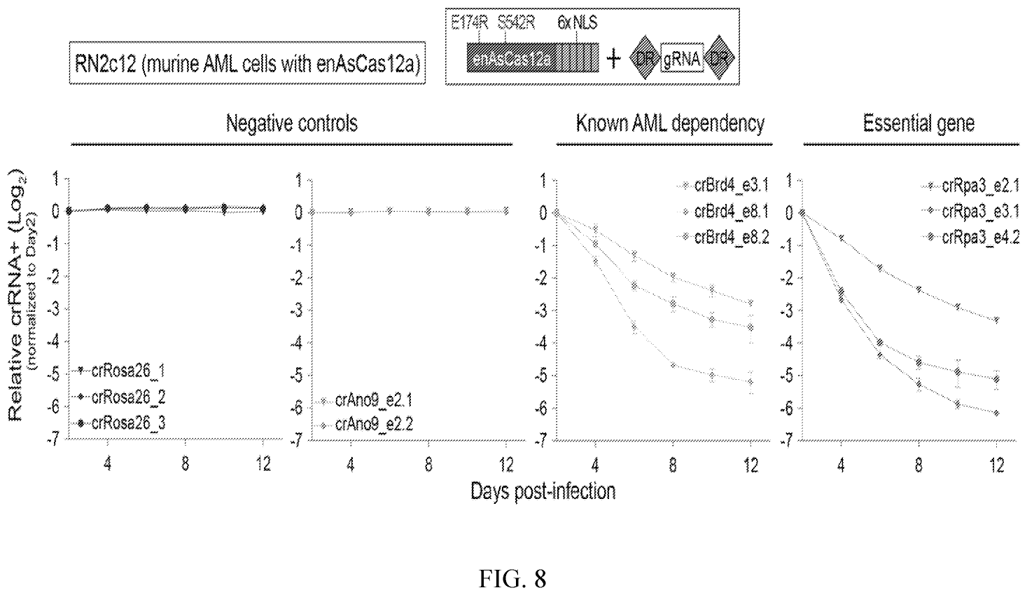

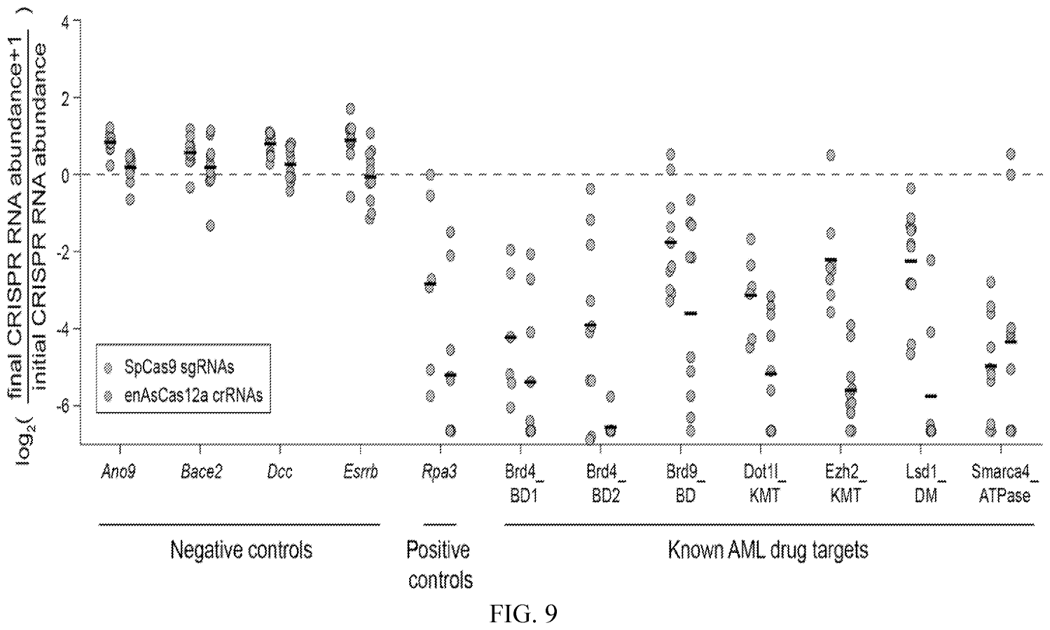

To benchmark the performance of the enAsCas12a system against SpCas9, a single-gene dropout screen was conducted in order to identify known cancer drug targets. The enAsCas12a- and SpCas9-based screens were performed in a murine Mll-A19/NrasG12D acute myeloid leukemia cell line (RN2), which has been previously used to identify epigenetic dependencies with SpCas9. To start, an enAsCas12a-expressing RN2 cell line (RN2c12) was created (FIG. 2A). The high knockout efficiency of the optimized enAsCas12a system was then verified using crRNAs targeting essential gene Rpa3 and known leukemia dependency Brd41 (FIG. 8). A customized enAsCas12a crRNA library was constructed targeting 155 protein domains involved in epigenetic regulation. Dropout screens in RN2 cells were performed using either this epigenetic-focused enAsCas12a crRNA library or a previously published SpCas9 sgRNA library (FIG. 2A). A CRISPR Score (CS), defined as the average log 2 fold-change of all crRNAs targeting a given protein domain, was used to quantify each domain's essentiality in supporting cancer cell proliferation (FIG. 2A). Spike-in positive and negative control CRISPR RNAs (enAsCas12a crRNA or SpCas9 sgRNA) included in the libraries validated the overall accuracy of both enAsCas12a and SpCas9 approaches (FIG. 9). Data from biological replicates of enAsCas12a screens were highly correlated with each other (r=0.96) as well as with the SpCas9 screen data (r=0.93) (FIG. 2B-2C). Notably, both screens revealed all known drug targets and dependencies in this murine Mll-Af9 leukemia model (FIG. 2B, 2C and FIG. 9). Collectively, these data support that the enAsCas12a system performs with similar robustness to SpCas9 in single-gene dropout screening.

### Example 3: enAsCas12-Based Combinatorial Knock-Out Screen Identifies Epigenetic Regulators

While single-gene knockout screens can capture a small subset of potential cancer drug targets, they miss synthetic sick/lethal genetic interactions that arise only from the simultaneous perturbation of two or more genes. It was next sought to take advantage of the multi-gene knockout capacity of enAsCas12a for combinatorial genetic screening. To evaluate the double knockout efficiency of enAsCas12a, cellular competition assays were performed in RN2c12 cells

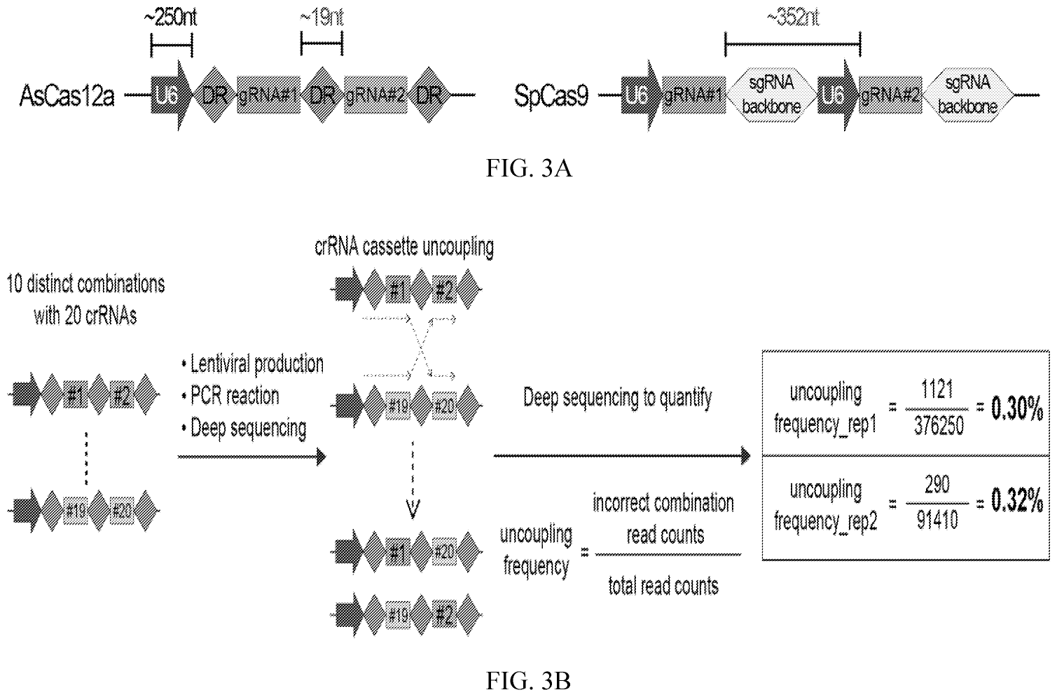

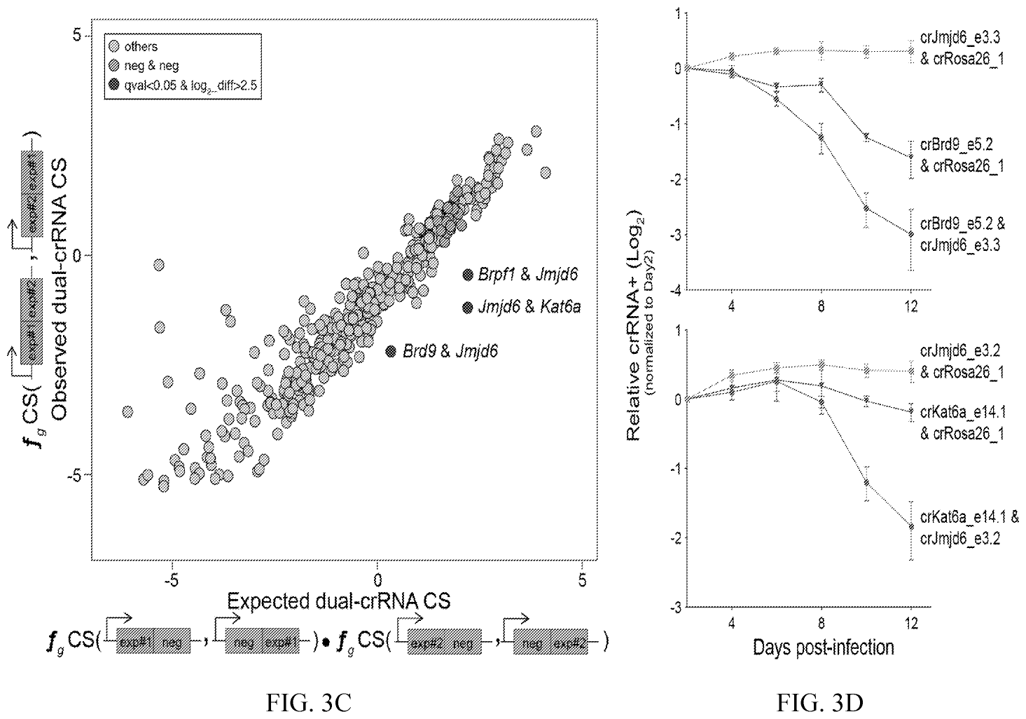

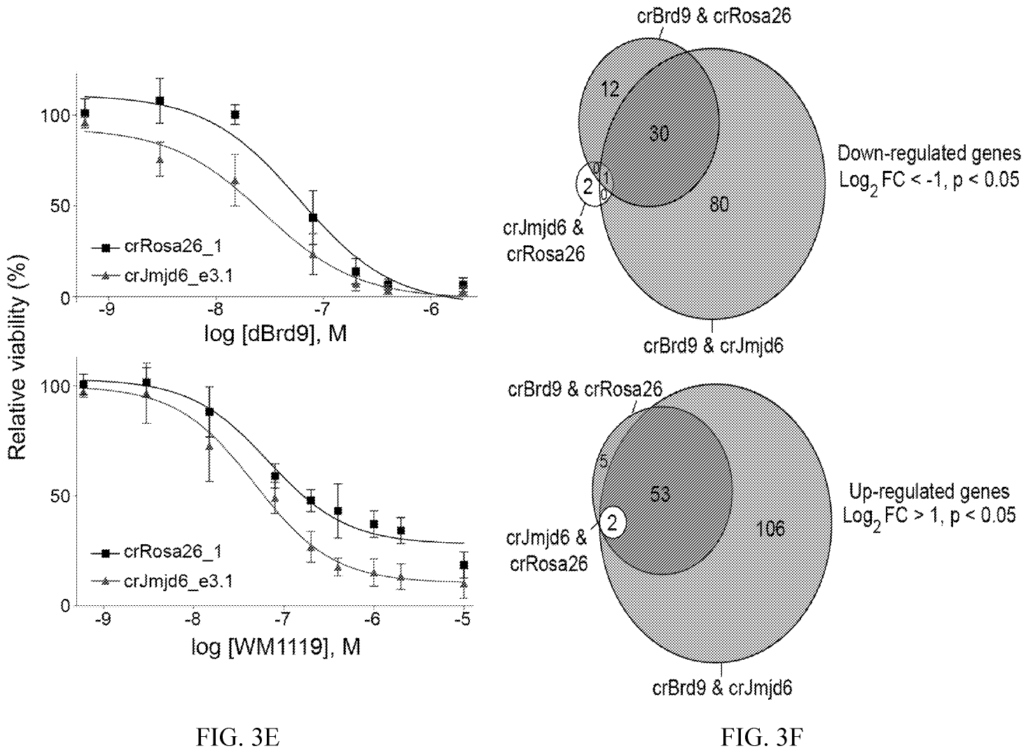

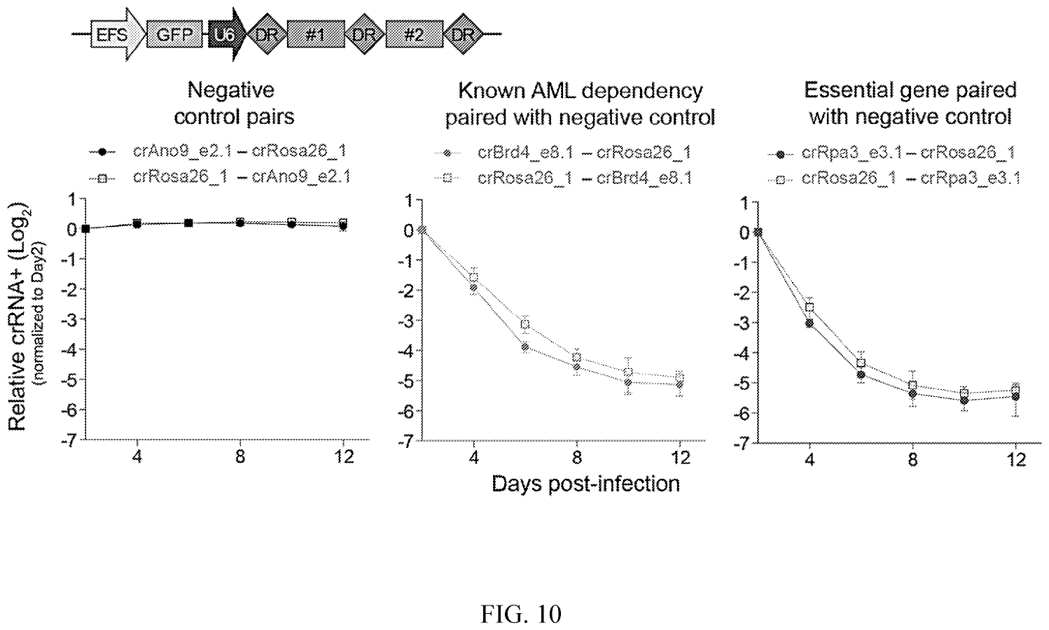

by expressing rearranged dual-crRNAs targeting essential genes and negative control loci under one U6 promoter (FIG. 3A and FIG. 10). It was confirmed that enAsCas12a can mutate targeted genes effectively using a dual-crRNA expression array. Observations also found that the phenotypic effect of a crRNA was biased slightly in favor of the first position 3′ to the U6 promoter (FIG. 10).

The high recombination frequency of DNA elements in a Cas9 dual-sgRNA expression vector largely restricts its application in combinatorial genetic screens. This recombination, which uncouples paired sgRNA-encoding DNA elements, can occur during lentiviral production, PCR amplification, and deep sequencing. Others have reported 10-40% sgRNA uncoupling in Cas9 systems. It was predicted that the recombination frequency would be significantly lower for AsCas12a, since the length of homologous sequence between two crRNAs is only 19-20 nt, compared to ˜352 nt in dual-sgRNA Cas9 systems (FIG. 3A). To measure the DNA uncoupling frequency in the enAsCas12a system, a mock genetic screen was performed with a library of unique dual-crRNAs. Observations found a DNA uncoupling frequency of ˜0.3% in the enAsCas12a dual-crRNA cassette (FIG. 3B), at least 35 times lower than that observed with Cas9.

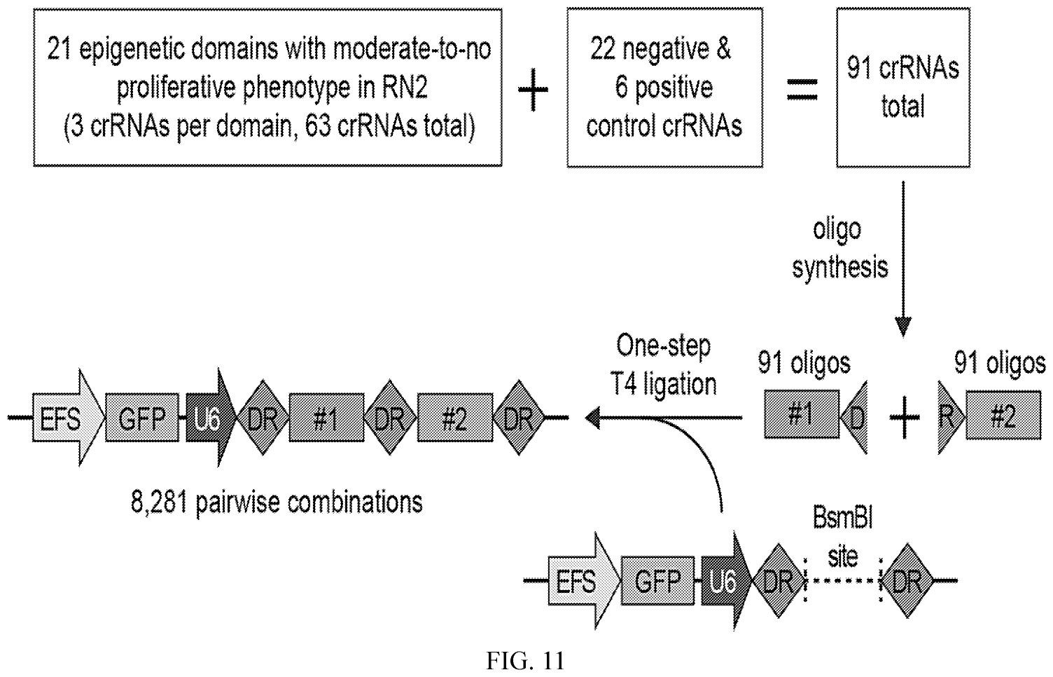

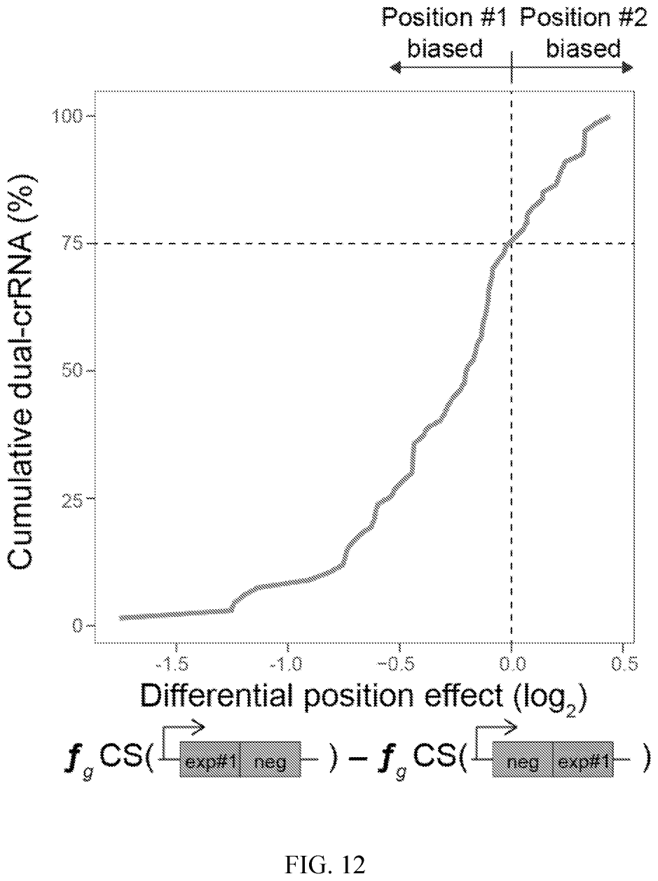

Next, it was evaluated how well enAsCas12a identifies synthetic sick/lethal genetic interactions in dual-crRNA dropout screening. 21 epigenetic regulatory domains were selected with moderate-to-no effect on proliferation from the single-crRNA enAsCas12a screen (FIG. 2B). A dual-crRNA library was then built with 8,281 pairwise combinations using a simple one-step ligation protocol (FIG. 11). A screen in RN2c12 cells was performed and quantified the abundance of crRNA pairs by directly deep sequencing the dual-crRNA expression cassette. As in the cellular competition assay (FIG. 10), observations found an apparent crRNA position effect during pooled library screening, where 75% of the experimental crRNA showed a stronger effect in the first position of a dual-crRNA array (FIG. 12). To account for this position bias, a Gaussian distribution model was used to adjust the dual-crRNA CS (/gCS). When an observed/gCS of two protein domains is significantly, at least two s.d., lower than the expected score from the combination of individual-domain /gCS, this domain pair is considered to have a synergistic interaction. Interestingly, the double knockout screen identified three unexpected synthetic sick interaction pairs, Brd9&Jmjd6, Jmjd6&Kat6a, and Brpf1&Jmjd6, which, when perturbed simultaneously, showed stronger proliferation inhibition (FIG. 3C). Jmjd6 appeared in all three pairs, suggesting that its perturbation might sensitize RN2 cells to various stresses.

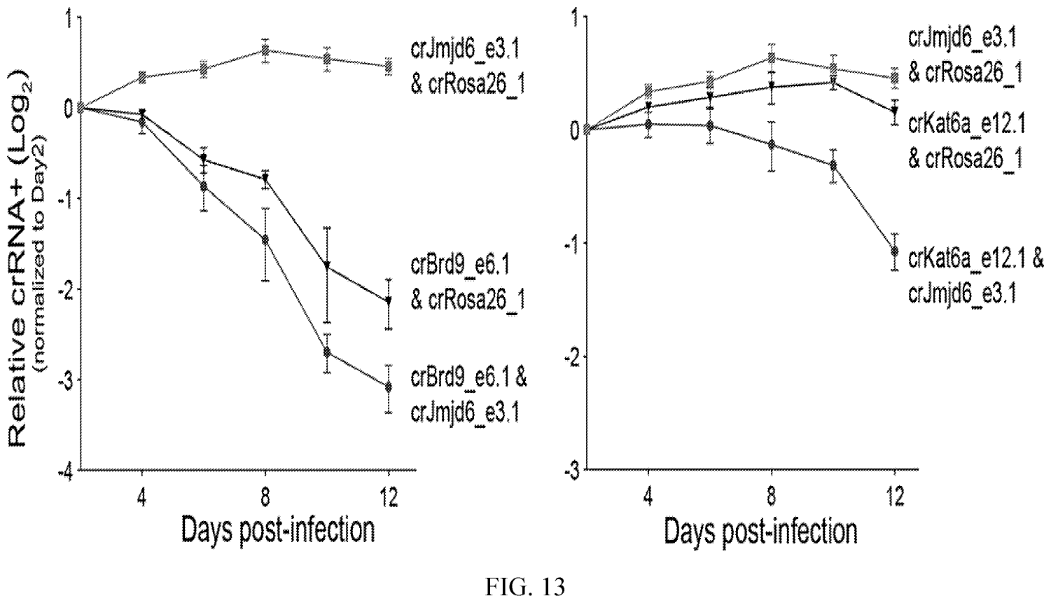

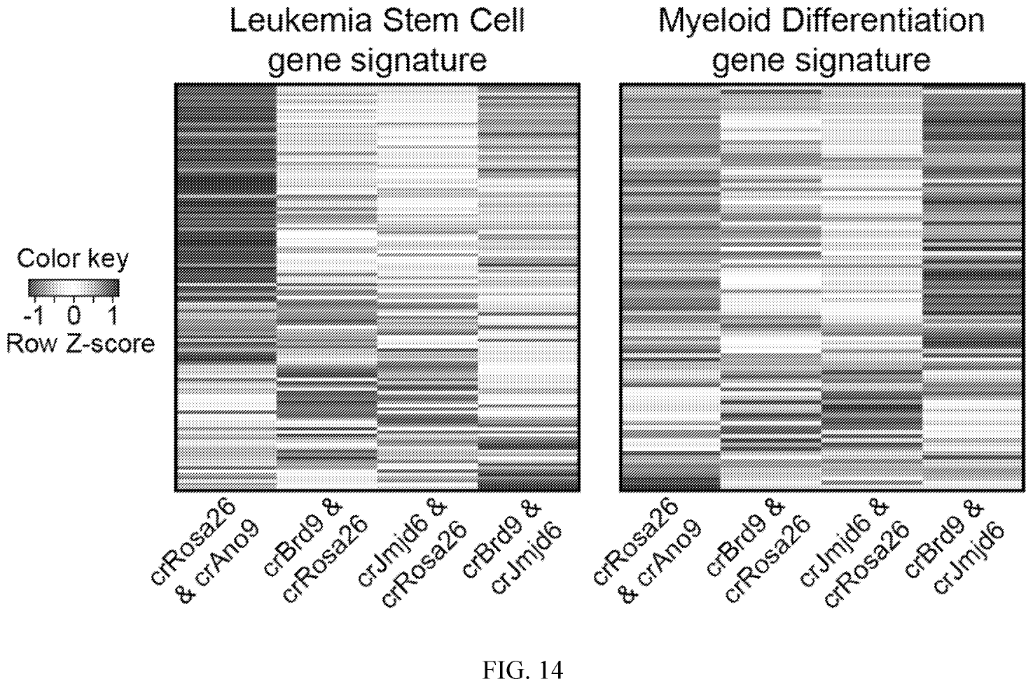

BRD9 participates in nucleosome remodeling, JMJD6 functions as a protein hydroxylase or histone demethylase, and KAT6A acts a histone acetyltransferase. Based on their known functions, it would not be obvious to predict synergistic interaction of any of these three genes in leukemia. Studies then proceeded to validate Brd9&Jmjd6 and Jmjd6&Kat6a interactions because BRD9 and KAT6A small molecule inhibitors are available. Using cellular competition assays, it was confirmed that dual-crRNA knockout of either Brd9&Jmjd6 or Jmjd6&Kat6a has a synergistic suppressive effect on RN2 proliferation (FIG. 3D and FIG. 13). Next, chemical inhibitors were used to further validate these two screen-identified hits. Since Jmjd6 crRNAs had a minimal effect on cellular proliferation (FIG. 3D), a stable Jmjd6-deficient RN2c12 cell line was generated and tested for sensitivity to either a BRD9 or KAT6A/B inhibitor. Consistent with the dual-crRNA perturbation data, Jmjd6 knockout sensitized RN2c12 cells to both BRD9 and KAT6A/B inhibitors (FIG. 3E). To further characterize the role of Brd9&Jmjd6 in leukemia, RNA-seq was performed to compare the global transcription changes upon either single or double genetic perturbation of Brd9 and Jmjd6. This analysis showed that double knockout of Brd9&Jmjd6 has a cooperative effect on transcription when compared with either single knockout (FIG. 3F). Gene set enrichment analysis revealed that double knockout of Brd9&Jmjd6 significantly influenced both leukemia stem cell and myeloid differentiation programs, which are essential regulatory programs for leukemia survival (FIG. 14). Together, these data demonstrate the robustness of the enAsCas12a system in combinatorial genetic screening for identifying synergistic genetic interactions.

### Example 4: Further Cas12a Gene Editing Efficiency Development

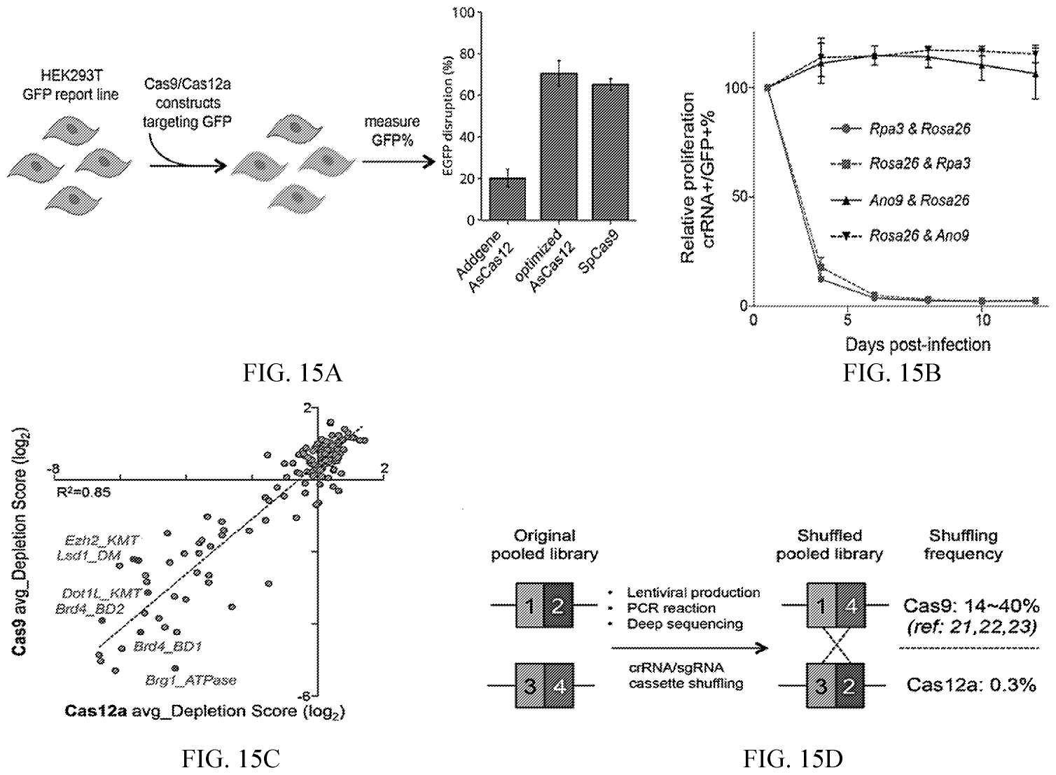

Accomplishments of the optimization: (i) increased the copy number of the nuclear localization sequence, (ii) stabilized the crRNA backbone with additional direct repeat hairpin structures, and (iii) introduced alterations to the Cas12a protein to increase its DNA binding affinity. Utilizing the improved Cas12a system and a HEK293T GFP reporter cell line, gene editing efficiencies comparable to Cas9 can be achieved (FIG. 15A), but with the distinctive advantage that the Cas12a system can mutate two genes simultaneously (FIG. 15B). To evaluate the performance of the engineered Cas12a system in loss-of-function pooled genetic screens, studies benchmarked it against Cas9. Specifically, drop-out genetic screens were performed targeting epigenetic regulators in RN2 cells, a murine AML, cell model with well-defined epigenetic dependencies at the single gene level. Cas12a performed as well as Cas9 in

genetic screens at the single gene level (FIG. 15C). Next a customized Cas12a double-crRNA library was constructed to assess the DNA shuffling effect of the crRNA pairs. Notably, data consistently demonstrated low DNA shuffling (˜0.3%) of the Cas12a double-crRNA cassette versus 14˜40% observed with Cas9 system. (FIG. 15D).

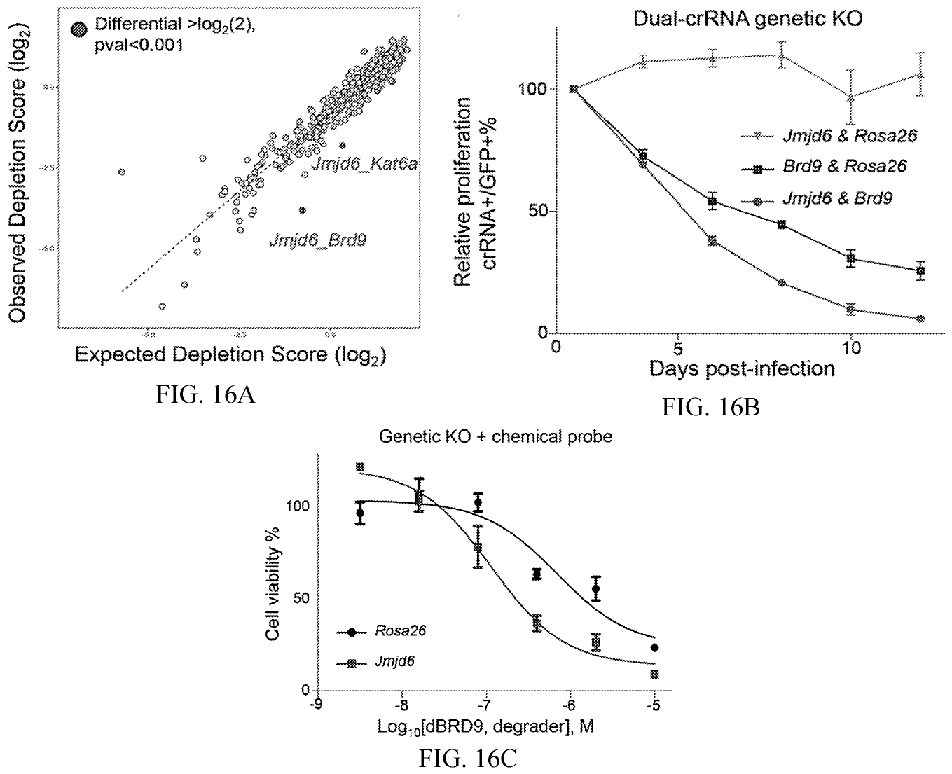

To demonstrate the performance of the engineered Cas12a system in double knockout genetic screening, drop-out genetic screens in RN2 cells were completed. Pairs of epigenetic regulators were identified that when perturbed at the same time show severe impact on cancer cell growth. First, a customized Cas12a-crRNA library was constructed targeting all possible pairs of 25 epigenetic regulators (3 crRNA per protein domain, with 16 negative control crRNAs, 8281 combinations total) and performed a pooled drop-out genetic screen in RN2 cells (FIG. 16A). Notably, it was observed that knockout of two gene pairs, Brd9/Jmjd6 and Kat6a/Jmjd6, significantly inhibited AML proliferation compared to individual gene knockout (FIG. 16A).

These results were further validated through cellular competition assays with individual crRNAs and small molecule inhibitors targeting either Brd9 or Kat6a (FIG. 16B-16C). The genetic interactions observed between Brd9/Jmjd6 and Kat6a/Jmjd6 gene pairs are unexpected, since the nucleosome remodeling (BRD9), histone demethylase (JMJD6), and histone acetyltransferase (KAT6A) activities are not predicted to function synergistically.

### Example 5: Unveiling Combinatorial Epigenetic Vulnerabilities in Cancer

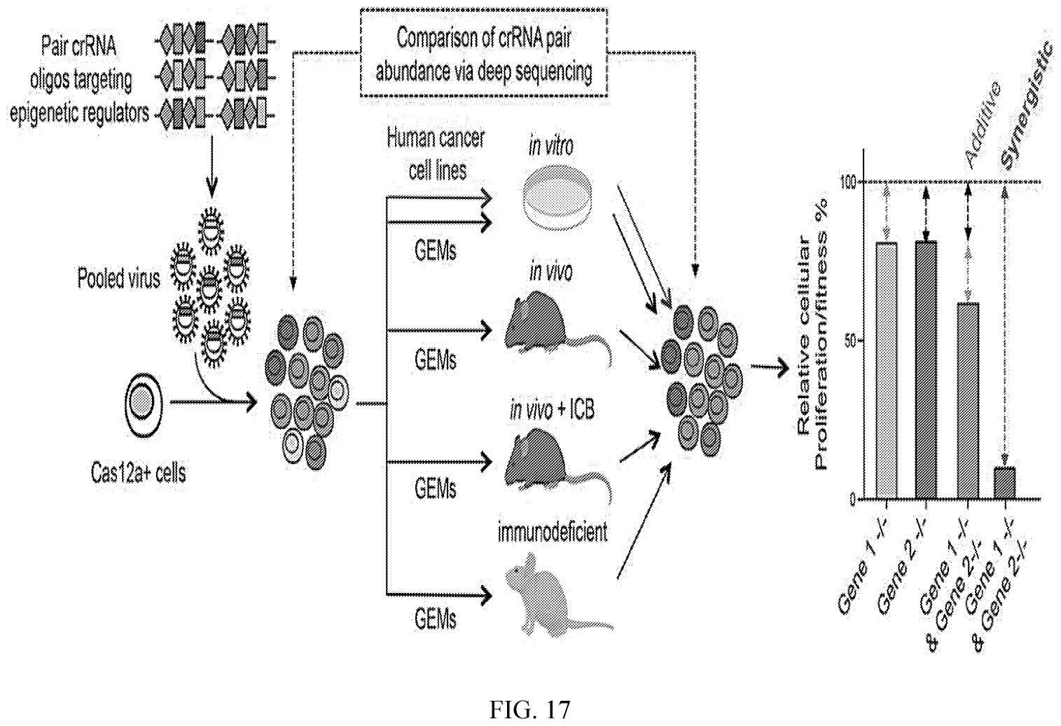

Unbiased loss-of-function genetic screens were performed in HCC to identify synthetic lethal and synergistic genetic interactions between two epigenetic regulators (FIG. 17). To enable a simple yet high-throughput genetic screening of mutagenizing two genes simultaneously, a bacterial immune defense system CRISPR-Cas12a was engineered for robust gene editing in mammalian cells. Cas12a has unique properties that make it an ideal tool for multi-gene targeting. Critically, while studies focused initially on epigenetic regulators in HCC, optimization of the CRISPR-Cas12a system for combinatorial genetic screens is widely applicable across diverse tumor types.

The overarching goal was to identify and elucidate new combinatorial epigenetic dependencies for HCC. Technological advances have expanded the repertoire of loss-of-function genetic approaches with the advent of CRISPR screening: a technology that employs a scalable and rapid, yet simple, programmable nuclease tool to construct genome-wide knockout libraries at known sites to screen for new disease vulnerabilities. Despite these advances, CRISPR-based genetic screening has yet to achieve its potential, as these systems currently do not permit simple and robust combinatorial screening for identification of synthetic lethal and synergistic genetic interactions. In conventional CRISPR screens, the Cas9 protein is guided by a single guide RNA (sgRNA) with a unique stretch of 20 nucleotides complementary to the target genomic DNA to generate a double strand DNA break. In the absence of a homology-directed repair DNA template, these breaks will be repaired through an error prone non-homology end joining pathway, which results in small insertion/deletion (indels) mutations around the break site. By directing the indels within a functionally relevant domain of a gene product, it has been shown that CRISPR-Cas9 can produce a higher fraction of null and hypomorphic mutations. This not only substantially increases the potency of genetic screening but also allows high-throughput identification of protein domains that are suitable drug targets in cancer because this method evaluates protein domain function directly from genetic screening. While the current CRISPR-Cas9 system can efficiently enable single-gene level loss-of-function genetic screening; it presents several major technical challenges for combinatorial genetic screening, which requires efficient knockout of two genes simultaneously in cells. For example, the long and repetitive DNA elements required to express dual Cas9 sgRNAs can cause severe DNA shuffling/swapping, creating technical challenges for pooled sgRNA cloning, deep sequencing, and data analysis. Hence, this inability to robustly and efficiently simultaneously mutagenize two genes in genomic screens limits the discovery of new synthetic lethal and synergistic genetic interactions that may provide novel therapeutic opportunity for disease treatment.

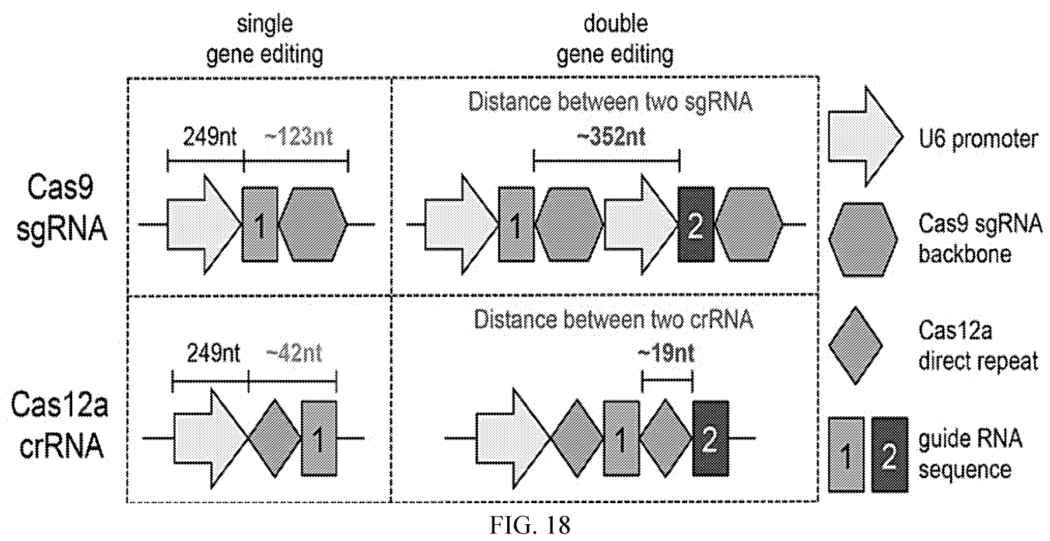

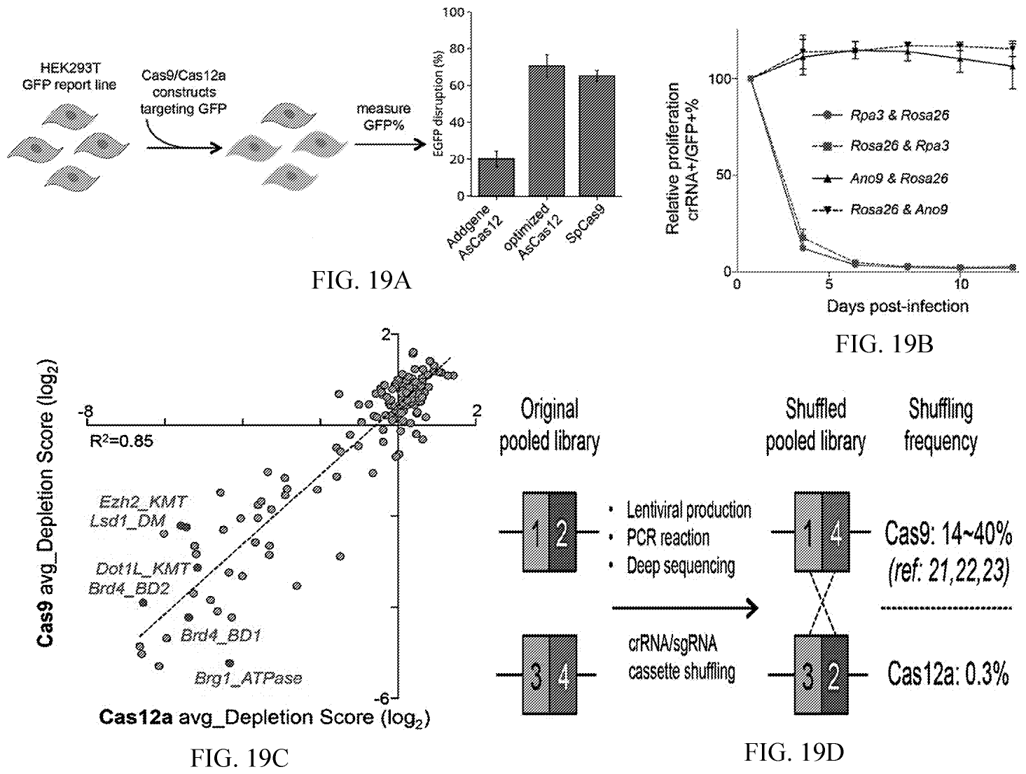

The scientific premise rests on the ability to develop a scalable, CRISPR-Cas12a approach to efficiently mutagenize two epigenetic regulators in a single cancer cell. A new CRISPR-Cas12a system for multiplexed gene editing in mammalian cells was developed. Similar to Cas9, Cas12a is a CRISPR RNA-guided DNA endonuclease. By reprogramming the CRISPR RNA (crRNA) sequence, Cas12a can be directed to mutagenize the designated target gene. The Cas12a system has unique features that make it an attractive platform for developing a multiplex gene editing toolkit (FIG. 18); First, Cas12a processes the crRNA array using its intrinsic RNase activity, allowing for simple multi-gene editing with a single crRNA array, and secondly, Cas12a uses a single crRNA for gene editing that is significantly shorter than a Cas9 sgRNA (˜42nt of Cas12a vs. ˜123nt of Cas9), making pooled CRISPR library construction much easier. It is well documented that two pieces of DNA elements will shuffle in a distance- and homology-dependent manner during virus production, PCR, and deep sequencing steps. This is a confounding technical limitation of double gene editing using the conventional Cas9 system. In contrast to the long distance (˜352nt) between two sgRNAs in the Cas9 system, the short sequence between two Cas12a crRNAs (˜19nt) effectively reduces DNA shuffling (FIG. 18 and FIG. 19D). Moreover, complex combinatorial Cas12a crRNA libraries targeting thousands of genes can be easily cloned through regular T4 ligation.

Despite their advantages, the publicly available Cas12a versions all have low gene editing efficiency in comparison to the commonly used Cas9 (FIG. 19A). To overcome this challenge, studies have advanced and optimized the Acidaminococcus sp Cas12a (referred to as Cas12a) system, resulting in marked improvement in gene editing efficiency. To this end studies have (i) increased the copy number of the nuclear localization sequence, (ii) stabilized the crRNA backbone with additional direct repeat hairpin structures, and (iii) introduced alterations to the Cas12a protein to increase its DNA binding affinity (based on the Cas12a crystal structure, PDB:5B43). Utilizing the improved Cas12a system and a HEK293T GFP reporter cell line, gene editing efficiencies comparable to Cas9 can be achieved (FIG. 19A), but with the distinctive advantage that the Cas12a system can mutate two genes simultaneously (FIG. 19B). To evaluate the performance of the engineered Cas12a system in loss-of-function pooled genetic screens, it was benchmarked against Cas9. Specifically, drop-out genetic screens were performed targeting epigenetic regulators in RN2 cells, a murine AML cell model with well-defined epigenetic dependencies at the single gene level. Cas12a performed as well as Cas9 in genetic screens at the single gene level (FIG. 19C). A customized Cas12a double-crRNA library was next constructed to assess the DNA shuffling effect of the crRNA pairs. Notably, the data consistently demonstrated low DNA shuffling (˜0.3%) of the Cas12a double-crRNA cassette versus 14˜40% observed with Cas9 systems. (FIG. 19D). Without wishing to be bound by theory, these findings indicated that the CRISPR RNA-guided Cas12a has unique features that allow efficient multi-gene editing which can be engineered and utilized in combinatorial genetic screens in human and mouse HCC cell lines to identify synthetic lethal and synergistic genetic interactions to inform new therapeutic targets.

### Example 6: Identification of Cell-Autonomous HCC Specific Combinatorial Epigenetic Dependencies

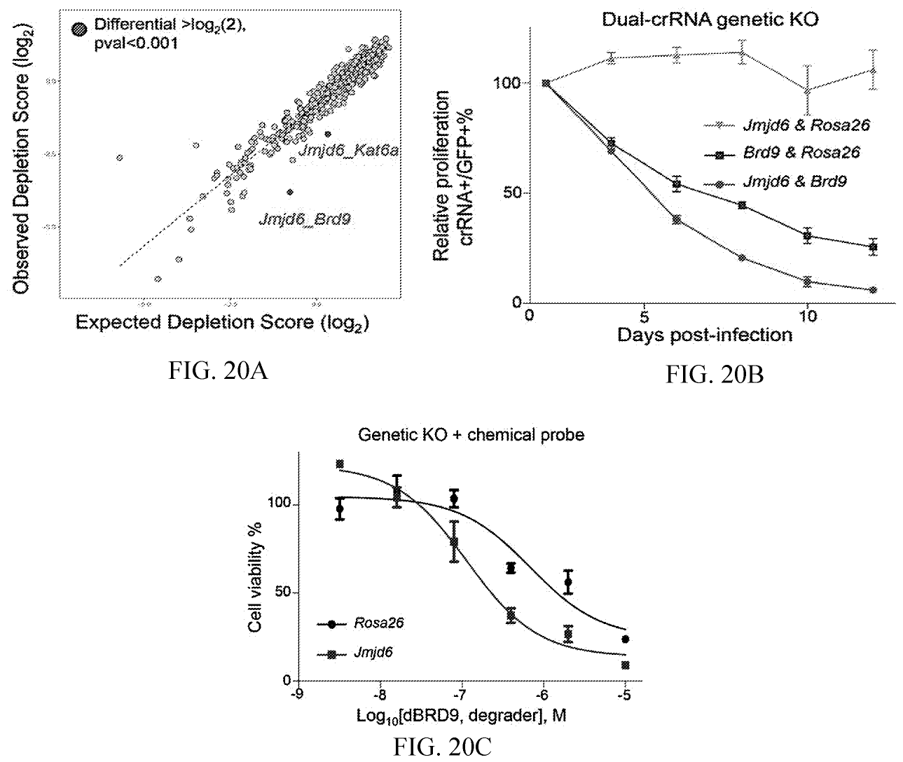

Alterations in transcription factors and epigenetic regulators are frequently observed in hepatocellular carcinoma (HCC), however, it is unknown whether these genetic alterations are driver mutations. It is also not known whether dysregulation of the epigenetic landscape and the transcriptional circuitry in HCC can create de novo epigenetic dependencies. At the level of a single genetic perturbation, loss-of-function genetic screens against epigenetic regulators performed in HCC yielded a surprisingly small number of epigenetic vulnerabilities, in contrast to the much larger number of vulnerabilities found in other malignancies, such as AML. Without wishing to be bound by theory, it can be hypothesized that single-gene level genetic screens are very likely missing critical epigenetic regulators in HCC. It can be postulated that this is due in part to the potential redundancy of epigenetic regulators, where one regulator can compensate for another to sustain critical epigenetic pathways when its homolog or paralog pathway is perturbed. Single-gene level genetic screens fail to reveal synthetic lethal and synergistic interactions between gene pairs. Hence, strong cancer epigenetic dependencies have yet to be discovered, despite the fact that data (see FIG. 20) strongly suggests that they are likely to exist.

To comprehensively annotate cell-autonomous epigenetic dependencies in HCC, loss-of-function combinatorial genetic screening was performed in HCC cells using the newly developed Cas12a platform. Data confirms that the optimized Cas12a system can efficiently knockout two genes simultaneously in both mouse and human cancer cell lines (FIG. 19B). To test the feasibility of double-knockout genetic screening, a customized crRNA library was created with all the possible pair combinations of 25 epigenetic regulators (3 crRNA per protein domain, with 16 negative control crRNAs, 8281 combinations total) and performed a pooled drop-out genetic screen in RN2 cells (FIG. 20A). Notably, it was observed that knockout of two gene pairs, Brd9/Jmjd6 and Kat6a/Jmjd6, significantly inhibited AML proliferation as compared to individual gene knockout (FIG. 20A). It was further validated these results through cellular competition assays with individual crRNAs and small molecule inhibitors targeting either Brd9 or Kat6a (FIGS. 4B-4C). The genetic interactions observed between Brd9/Jmjd6 and Kat6a/Jmjd6 gene pairs are unexpected, since the nucleosome remodeling (BRD9), histone demethylase (JMJD6), and histone acetyltransferase (KAT6A) activities are not predicted to function synergistically. Without wishing to be bound by theory, these data support the feasibility of performing unbiased combinatorial genetic screens and highlight the possibility of identifying new and unexpected synergistic regulatory pathways for therapeutic intervention.

### Example 7: Identification of Non-Cell-Autonomous HCC Specific Combinatorial Epigenetic Dependencies

Genetic alterations in cancer not only reprogram the cell into an oncogenic state but also alter its interaction with cells in the local environment allowing escape from host immunosurveillance. By modulating the communication between tumor cells and immune cells, the host immune system can be reactivated to attack the tumor cells. Immune checkpoint blockade (ICB) therapy has shown notable clinical success in certain tumor types by blocking the interaction of two immune checkpoint proteins PD-L1, in tumor cells, and PD-1, in immune cells. However, as single-agent therapy, ICB has limited efficacy in many tumor types, including HCC. The cancer patient genome sequencing project confirmed the immunosuppressive environment of HCC with few infiltrating anti-tumor immune cells and high expression of immune checkpoint proteins in the tumor. Recent studies suggest that manipulating epigenetic programs in tumor cells can increase anti-tumor immune responses through reactivating human endogenous retroviruses, cancer-testis antigens, and neo-antigens. Since epigenetic regulators are frequently altered in HCC, it was hypothesized that epigenetic pathways in the tumor cells are rewired to suppress host immunosurveillance. To comprehensively identify non-cell-autonomous epigenetic regulators that maintain an immunosuppressive state in HCC, combinatorial loss-of-function in vivo genetic screening was performed using a mouse model of HCC.

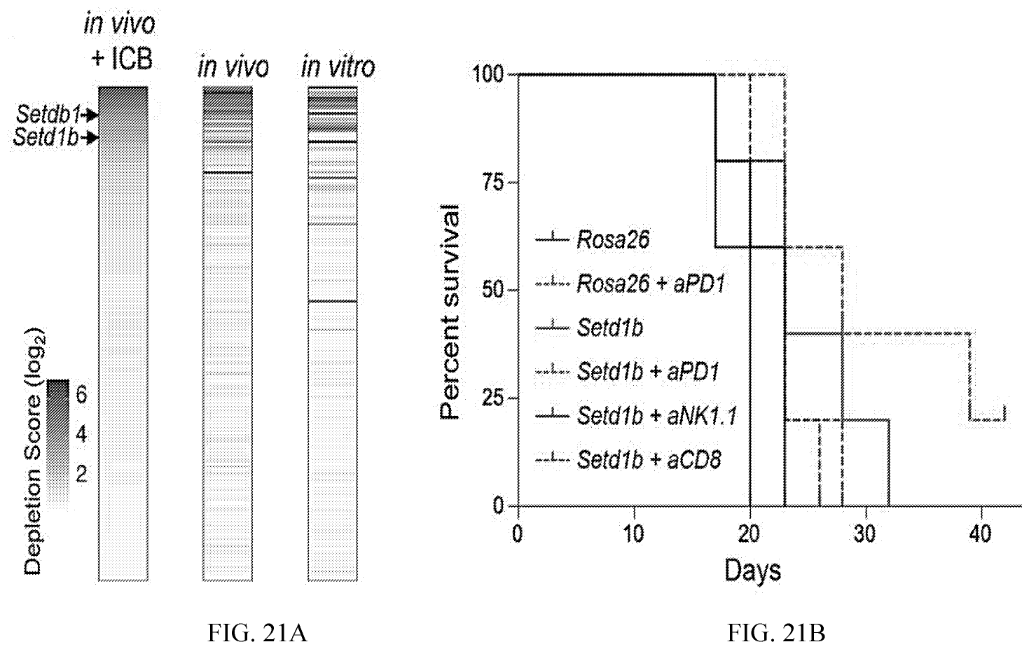

To demonstrate the feasibility of identifying epigenetic regulators that modulate the cancer immune microenvironment, several murine tumor models with high CRISPR Cas9-editing efficiency for in vivo protein domain-based genetic screening were developed. A domain-focused Cas9 sgRNA library targeting epigenetic enzymes was created and used to perform three types of genetic screens in a murine melanoma model (B16F10): (i) in vitro, (ii) in vivo, and (iii) in vivo with a PD-1 inhibitor used for ICB therapy. Notably, these single-gene level Cas9 loss-of-function genetic screens identified two histone methyltransferase genes, Setd1b and Setdb1, as non-cell-autonomous dependencies of B16F10 melanoma. These two genes were required for B16F10 melanoma cell proliferation and survival in vivo, but not in cell culture (FIG. 21A), consistent with a non-cell autonomous mechanism. Genetic knockout of Setd1b in B16F10 melanoma cells revealed increased infiltration of NK and cytotoxic CD8 T cells in tumors generated by inoculation of B16F10ΔSETD1B melanoma cells into syngeneic wild-type C57Bl/6 mice, and immunoprofiling by flow cytometry. Furthermore, antibody-mediated depletion of either NK or T cells in B16F10ΔSETD1B tumor bearing mice partially alleviated the inhibition of B16F10ΔSETD1B tumor growth (FIG. 21B). Together, without wishing to be bound by theory, these data support the scientific premise that in vivo loss-of-function genetic screens can identify tumor non-cell-autonomous dependencies.

### Example 8: Conclusion

Collectively, gene knockout efficiency of CRISPR-AsCas12a was optimized in mammalian cells by 1) increasing the number of NLS, 2) introducing an additional crRNA DR, and 3) employing an enhanced enAsCas12a variant (E174R/S542R). These improvements in AsCas12a are likely to be applicable to orthogonal Cas12a systems and to be beneficial for Cas12a-based base editors. It was shown that construction of an enAsCas12a dual-crRNA library requires only a simple one-step T4 ligation cloning reaction and results in a low recombination frequency dual crRNA library for genetic screening. Through double knockout screening, unexpected synergistic epigenetic vulnerabilities in Mll-Af9 leukemia cells were identified and validated, demonstrating the power of this method. Together, these studies developed high-performance AsCas12a-based approaches for unbiased, combinatorial genetic screening, which may be applicable to many 165 cancer types and disease models for therapeutic target discovery.

### Enumerated Embodiments

The following enumerated embodiments are provided, the numbering of which is not to be construed as designating levels of importance.

Embodiment 1 provides a method of combinatorial genetic screening in a cancer cell, the method comprising administering to the cancer cell a CRISPR-Cas12a system, wherein the CRISPR-Cas12a system comprises an enhanced Cas12a variant, a modified nuclear localization signal (NLS), and a modified dual direct repeat CRISPR RNA (crRNA), whereby the CRISPR-Cas12a system mutates multiple genomic regions simultaneously, and combinatorial genetic screening is achieved.

Embodiment 2 provides the method of embodiment 1, wherein the enhanced Cas12a variant is an Acidaminococcus Cas12a (AsCas12a) variant.

Embodiment 3 provides the method of embodiment 2, wherein the AsCas12a comprises a E174R mutation and/or a S542R mutation.

Embodiment 4 provides the method of any one of embodiments 1-3, wherein the enhanced Cas12a variant has increased DNA binding affinity and activity.

Embodiment 5 provides the method of embodiment 1, wherein the modified NLS comprises six copies of a NLS.

Embodiment 6 provides the method of embodiment 1, wherein the modified crRNA comprises from 3′ to 5′ a first direct repeat (DR) sequence, a gRNA, and a second direct repeat sequence.

Embodiment 7 provides the method of embodiment 1, wherein the crRNA comprises from 3′ to 5′ a first direct repeat sequence, a first gRNA, a second direct repeat sequence, a second gRNA, and a third direct repeat sequence.

Embodiment 8 provides the method of any one of embodiments 5-7, wherein any one of the direct repeats is about 19 nucleotides in length.

Embodiment 9 provides the method of embodiment 1, wherein the screening identifies genomic regions involved in cancer pathogenesis.

Embodiment 10 provides the method of embodiment 1, wherein the screening identifies epigenetic interactions in the cancer cell.

Embodiment 11 provides the method of embodiment 10, wherein the epigenetic interactions are synthetic sick/lethal interactions.

Embodiment 12 provides the method of embodiment 1, further comprising designing a cancer treatment based on the screening results.

Embodiment 13 provides the method of embodiment 1, further comprising a crRNA library, wherein the crRNA library comprises a plurality of crRNAs targeting a plurality of genomic regions involved in epigenetic regulation.

Embodiment 14 provides the method of claim 13, wherein each crRNA in the crRNA library comprises from 3′ to 5′ a first direct repeat sequence, a first gRNA, and a second direct repeat sequence.

Embodiment 15 provides the method of embodiment 13, wherein each crRNA in the crRNA library comprises from 3′ to 5′ a first direct repeat sequence, a first gRNA, a second direct repeat sequence, a second gRNA, and a third direct repeat sequence.

Embodiment 16 provides a method of identifying a combination of drug targets wherein a therapeutically synergistic effect is elicited when the combination of targets is treated, the method comprising: administering to a cell a composition comprising a CRISPR-Cas12 system, wherein the CRISPR-Cas12a system comprises an enhanced Cas12a variant, a modified nuclear localization signal (NLS), and a modified dual direct repeat CRISPR RNA (crRNA), whereby the CRISPR-Cas12a system mutates multiple genomic regions simultaneously, thereby identifying the drug target combination.

Embodiment 17 provides a method of treating cancer in a subject in need thereof, the method comprising: administering a CRISPR-Cas12a system to a cancer cell from the subject,

wherein the CRISPR-Cas12a system comprises an enhanced Cas12a variant, a modified nuclear localization signal (NLS), and a modified dual direct repeat CRISPR RNA (crRNA), whereby the CRISPR-Cas12a system mutates multiple genomic regions simultaneously, and combinatorial genetic screening is performed, determining a cancer treatment based on the screening results, and administering the cancer treatment to the subject.

Embodiment 18 provides the method of embodiment 16 or 17, wherein the enhanced Cas12a variant is an Acidaminococcus Cas12a (AsCas12a) variant.

Embodiment 19 provides the method of embodiment 18, wherein the AsCas12a comprises a E174R mutation and/or a S542R mutation.

Embodiment 20 provides the method of any one of embodiments 16-19, wherein the enhanced Cas12a variant has increased DNA binding affinity and activity.

Embodiment 21 provides the method of embodiment 16 or 17, wherein the modified NLS comprises six copies of a NLS.

Embodiment 22 provides the method of embodiment 16 or 17, wherein the modified crRNA comprises from 3′ to 5′ a first direct repeat (DR) sequence, a gRNA, and a second direct repeat sequence.

Embodiment 23 provides the method of embodiment 22, wherein the crRNA comprises from 3′ to 5′ a first direct repeat sequence, a first gRNA, a second direct repeat sequence, a second gRNA, and a third direct repeat sequence.

Embodiment 24 provides the method of any one of embodiments 16-23, wherein any one of the direct repeats is about 19 nucleotides in length.

Embodiment 25 provides the method of embodiment 17, wherein the screening identifies epigenetic interactions in the cancer cell.

Embodiment 26 provides the method of embodiment 25, wherein the epigenetic interactions are synthetic sick/lethal interactions.

Embodiment 27 provides the method of embodiment 16 or 17, further comprising a crRNA library, wherein the crRNA library comprises a plurality of crRNAs targeting a plurality of genomic regions involved in epigenetic regulation.

Embodiment 28 provides the method of embodiment 27, wherein each crRNA in the crRNA library comprises from 3′ to 5′ a first direct repeat sequence, a first gRNA, and a second direct repeat sequence.

Embodiment 29 provides the method of embodiment 27, wherein each crRNA in the crRNA library comprises from 3′ to 5′ a first direct repeat sequence, a first gRNA, a second direct repeat sequence, a second gRNA, and a third direct repeat sequence.

Embodiment 30 provides a composition comprising a first nucleic acid comprising a nucleotide sequence encoding an AsCas12a comprising a E174R mutation and a S542R mutation, and six nuclear localization sequences, and a second nucleic acid comprising a crRNA comprising a first direct repeat sequence, a first gRNA, and a second direct repeat sequence.

Embodiment 31 provides a composition comprising a first nucleic acid comprising a nucleotide sequence encoding an AsCas12a comprising a E174R mutation and a S542R mutation, and six nuclear localization sequences, and a second nucleic acid comprising a crRNA comprising a first direct repeat sequence, a first gRNA, a second direct repeat sequence, a second gRNA, and a third direct repeat sequence.

Embodiment 32 provides a composition comprising a first nucleic acid comprising a nucleotide sequence encoding an AsCas12a comprising a E174R mutation and a S542R mutation, and six nuclear localization sequences, and a crRNA library, wherein each crRNA in the crRNA library comprises from 3′ to 5′ a first direct repeat sequence, a first gRNA, a second direct repeat sequence, a second gRNA, and a third direct repeat sequence.

Embodiment 33 provides a kit useful for combinatorial genetic screening comprising any one of the compositions of embodiments 30-32.

Embodiment 34 provides a method of treating cancer in a subject in need thereof, the method comprising administering to the subject an inhibitor of Brd9 and an inhibitor of Jmjd6.

Embodiment 35 provides a method of treating cancer in a subject in need thereof, the method comprising administering to the subject an inhibitor Jmjd6 and an inhibitor of Kat6a.

Embodiment 36 provides the method of embodiments 34 or 35, wherein the cancer is leukemia.

Embodiment 37 provides the method of any one of embodiments 34-36, wherein the inhibitor is selected from the group consisting of a small molecule, an antibody, a CRISPR system, a miRNA, a drug, an inhibitory RNA, or a genome editing tool.

### Other Embodiments

The recitation of a listing of elements in any definition of a variable herein includes definitions of that variable as any single element or combination (or sub combination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of combinatorial genetic screening in a cancer cell, the method comprising administering to the cancer cell a CRISPR-Cas12a system,
wherein the CRISPR-Cas12a system comprises an enhanced Cas12a variant, a modified nuclear localization signal (NLS), and a modified dual direct repeat CRISPR RNA (crRNA),
whereby the CRISPR-Cas12a system mutates multiple genomic regions simultaneously, and combinatorial genetic screening is achieved.

2. The method of claim 1, wherein the enhanced Cas12a variant is an Acidaminococcus Cas12a (AsCas12a) variant.

3. The method of claim 2, wherein the AsCas12a comprises a E174R mutation and/or a S542R mutation.

4. The method of claim 1, wherein the enhanced Cas12a variant has increased DNA binding affinity and activity.

5. The method of claim 1, wherein the modified NLS comprises six copies of a NLS.

6. The method of claim 1, wherein the modified crRNA comprises from 3′ to 5′ a first direct repeat (DR) sequence, a first gRNA, and a second direct repeat sequence.

7. The method of claim 6, wherein the modified crRNA further comprises from 3′ to 5′ a second gRNA, and a third direct repeat sequence, wherein the second gRNA and third direct repeat sequence are located 5′ of the second direct repeat sequence.

8. The method of claim 6, wherein any one of the direct repeats is about 19 nucleotides in length.

9. The method of claim 1, wherein the screening identifies genomic regions involved in cancer pathogenesis.

10. The method of claim 1, wherein the screening identifies epigenetic interactions in the cancer cell.

11. The method of claim 10, wherein the epigenetic interactions are synthetic sick/lethal interactions.

12. The method of claim 1, further comprising designing a cancer treatment based on the screening results.

13. The method of claim 1, further comprising a crRNA library, wherein the crRNA library comprises a plurality of crRNAs targeting a plurality of genomic regions involved in epigenetic regulation.

14. The method of claim 13, wherein each crRNA in the crRNA library comprises from 3′ to 5′ a first direct repeat sequence, a first gRNA, and a second direct repeat sequence.

15. The method of claim 13, wherein each crRNA in the crRNA library comprises from 3′ to 5′ a first direct repeat sequence, a first gRNA, a second direct repeat sequence, a second gRNA, and a third direct repeat sequence.

16. A method of identifying a combination of drug targets wherein a therapeutically synergistic effect is elicited when the combination of targets is treated, the method comprising:
administering to a cell a composition comprising a CRISPR-Cas12 system, wherein the CRISPR-Cas12a system comprises an enhanced Cas12a variant, a modified nuclear localization signal (NLS), and a modified dual direct repeat CRISPR RNA (crRNA),
whereby the CRISPR-Cas12a system mutates multiple genomic regions simultaneously, thereby identifying the drug target combination.

17. A method of treating cancer in a subject in need thereof, the method comprising:
administering a CRISPR-Cas12a system to a cancer cell from the subject,
wherein the CRISPR-Cas12a system comprises an enhanced Cas12a variant, a modified nuclear localization signal (NLS), and a modified dual direct repeat CRISPR RNA (crRNA),
whereby the CRISPR-Cas12a system mutates multiple genomic regions simultaneously, and combinatorial genetic screening is performed,
determining a cancer treatment based on the screening results, and
administering the cancer treatment to the subject.

18. The method of claim 17, wherein the enhanced Cas12a variant is an Acidaminococcus Cas12a (AsCas12a) variant.

19. The method of claim 18, wherein the AsCas12a comprises a E174R mutation and/or a S542R mutation.

20. The method of claim 17, wherein the enhanced Cas12a variant has increased DNA binding affinity and activity.

21. The method of claim 17, wherein the modified NLS comprises six copies of a NLS.

22. The method of claim 17, wherein the modified crRNA comprises from 3′ to 5′ a first direct repeat (DR) sequence, a gRNA, and a second direct repeat sequence.

23. The method of claim 22, wherein the modified crRNA further comprises from 3′ to 5′ a second gRNA, and a third direct repeat sequence, wherein the second gRNA and third direct repeat sequence are located 5′ of the second direct repeat sequence.

24. The method of claim 22, wherein any one of the direct repeats is about 19 nucleotides in length.

25. The method of claim 17, wherein the screening identifies epigenetic interactions in the cancer cell.

26. The method of claim 25, wherein the epigenetic interactions are synthetic sick/lethal interactions.

27. The method of claim 17, further comprising a crRNA library, wherein the crRNA library comprises a plurality of crRNAs targeting a plurality of genomic regions involved in epigenetic regulation.

28. The method of claim 27, wherein each crRNA in the crRNA library comprises from 3′ to 5′ a first direct repeat sequence, a first gRNA, and a second direct repeat sequence.

29. The method of claim 27, wherein each crRNA in the crRNA library comprises from 3′ to 5′ a first direct repeat sequence, a first gRNA, a second direct repeat sequence, a second gRNA, and a third direct repeat sequence.

30. A composition comprising a first nucleic acid comprising a nucleotide sequence encoding an AsCas12a comprising a E174R mutation and a S542R mutation, and six nuclear localization sequences, and a second nucleic acid comprising one or more crRNAs comprising a first direct repeat sequence, a first gRNA, and a second direct repeat sequence.

31. The composition of claim 30, wherein the one or more crRNAs further comprise a second gRNA, and a third direct repeat sequence.

32. The composition of claim 30, wherein the one or more crRNAs comprise a crRNA library.

33. A kit useful for combinatorial genetic screening comprising the composition of claim 30.

34. (canceled)

35. A method of treating cancer in a subject in need thereof, the method comprising administering to the subject an inhibitor Jmjd6 and an inhibitor of Kat6a or Brd9.

36. The method of claim 35, wherein the cancer is leukemia.

37. The method of claim 35, wherein the inhibitor is selected from the group consisting of a small molecule, an antibody, a CRISPR system, a miRNA, a drug, an inhibitory RNA, or a genome editing tool.


SEQUENCE LISTING (4 sequences)

SEQ ID NO: 1 - protein, 1307 aa, Artificial Sequence
  AsCas12a
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 2 - protein, 1264 aa, Artificial Sequence
  MbCas12a
MLFQDFTHLYPLSKTVRFELKPIGKTLEHIHAKNFLSQDETMADMYQKVKAILDDYHRDFITKMMSEVTLTKLPEFYEVYLALRKNPKDDTLQKQLTEIQTALREEVVKPIDSGGKYKAGYERLFGAKLFKDGKELGDLAKFVIAQEGESSPKLPQIAHFEKFSTYFTGFHDNRKNMYSSDDKHTAIAYRLIHENLPRFIDNLQILVTIKQKHSVLYDQIVNELNANGLDVSLASHLDGYHKLLTQEGITAYNTLLGGISGEAGSRKIQGINELINIHHNQHCQKSERIAKLRPLHKQILSDGMGVSFLPSKFADDSEVCQAVNEFYRHYAHVFAKVQSLFDRFDDYRKDGIYVEHKNLNELSKQAFGDFALLGRVLDGYYVDVVNPEFNERFAKAKTDNAKEKLTKEKDKFIKGVHSLASLEQAIEHYIAGHDDESVQAGKLGQYFKHGLAGVDNPIQKIHNSHSTIKGFLERERPAGERALPKIKSDKSPEMTQLRQLKELLDNALNVVHFAKLLTTKTTLDNQDGNFYGEFGALYDELAKIATLYNKVRDYLSQKPFSTEKYKLNFGNPTLLNGWDLNKEKDNFGVILQKDGCYYLALLDKAHKKVFDNAPNAGKSVYQKMVYKLLPGPNKMLPKVFFAKSNLDYYNPSAELLDKYAQGTHKKGNNFNLKDCHALIDFFKASINKHPEWQHFGFEFSPTSSYQDLSDFYREVEPQGYQVKFVDINAEYIDELVEQGQLYLFQIYNKDFSPKAHGKPNLHTLYFKALFSENNLANPIYKLNGEAEIFYRKASLDMNETTIHRAGEVLENKNPDNPKKRQFVYDIIKDKRYTQDKFMLHVPITMNFGVQGMTIKEFNKKVNQSIQQYNDVNVIGIDRGERHLLYLTVINSKGEILEQRSLNDIITTSANGTQMTTPYHKILDKREIERLNARVGWGEIETIKELKSGYLSHVVHQISQLMLKYNAIVVLEDLNFGFKRGRFKVEKQIYQNFENALIKKLNHLALKDKADDEIGSYKNALQLTNNFTDLKSIGKQTGFLFYVPAWNTSKIDPATGFVDLLKPRYENIAQSQAFFGKFDKIFYNADKGYFEFYVDYAKFTDKAKNSRQTWVICSHGDKRYVYDKTANQNKGATIGINVNDELKSLFARYHINDKQPNLVMDICQNNDKEFHKSLMYLLKALLALRYSNASSDEDFILSPVADDKGVFFNSALADDTQPQNADANGAYHIALKGLWLLNELKNSNDLDKIKLAIDNQTWLNFAQNR

SEQ ID NO: 3 - protein, 1228 aa, Artificial Sequence
  LbCas12a
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 4 - protein, 1300 aa, Artificial Sequence
  FnCas12a
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN